(19) United States
(12) Patent Application Publication
(10) Pub. No.: US 2018/0280951 A1
(43) Pub. Date: Oct. 4, 2018

(54) Process for the Oligomerisation of Olefins by Coordinative Chain Transfer Polymerisation

(71) Applicant: Sasol Performance chemicals GmbH, Hamburg (DE)

(72) Inventors: Albert Boddien, Itzehoe (DE); Rhett Kempe, Bayreuth (DE); Winfried Kretschmer, Weidenberg (DE); Andreas Gollwitzer, Bayreuth (DE)

(21) Appl. No.: 15/571,315

(22) PCT Filed: May 13, 2016
(86) PCT No.: PCT/EP2016/000789

§ 371 Date: Nov. 2, 2017

(30) Foreign Application Priority Data
May 13, 2015 (EP) 15167707.7
Jul. 21, 2015 (GB) 1512872.1

(51) Int. Cl. B01J 31/18; B01J 31/14; C07C 2/88

(52) U.S. Cl. CPC B01J 31/1805; B01J 31/143; C07C 2531/22; B01J 2531/48; C07C 2/88

(57) ABSTRACT

The present invention relates to a process for the oligomerisation of olefins, in particular ethylene, via coordinative chain transfer polymerisation (CCTP) and alkyl elimation reaction. A preferred embodiment of the invention relates to CCTP of olefins, in particular ethylene, with the use of guanidinato, amidinato or hydrocarbyl-2-pyridyl amine complexes of titanium, zirconium or lanthanides, a nickel or cobalt compound as chain displacement catalyst (CDC) and one or more chain shuttling agents (CSA) such as a main group metal alkyl.

### INTRODUCTION

The present invention relates to a process for the oligomerisation of olefins, in particular ethylene, via coordinative chain transfer polymerisation (CCTP) and alkyl elimination reaction. A preferred embodiment of the invention relates to CCTP of olefins, in particular ethylene, with the use of guanidinato, amidinato or hydrocarbyl-2-pyridyl amine complexes of titanium, zirconium or lanthanides, a chain displacement catalyst (CDC) being a nickel or cobalt compound, and one or more chain shuttling agents (CSA), such as dihydrocarbyl zinc or trihydrocarbyl aluminium or both. The characteristics of the process according to the invention are that various olefins can be produced using only catalytic amounts of CSA. Further by changing the parameters of the process oligomerized olefins having a Schulz-Flory or Gauss or Poisson distribution can be obtained as wanted.

### BACKGROUND OF THE INVENTION AND PRIOR ART DISCUSSION

The oligomerisation of olefins can yield product distributions with regard to chain lengths which are either Gauss or Poisson distributions or Schulz-Flory distributions. A Gauss or Poisson distribution is characterised by the formula Xₚ=(x^{p}·e^{-x})/p!, and a Schulz-Flory distribution by the formula Xₚ=β(1+β)^{-p}, whereas Xₚ is the mole fraction with p added olefins, x is the Gauss or Poisson distribution coefficient equal to the average number of olefin molecules added per M-C bond, and ß is the Schulz-Flory distribution coefficient. A Gauss or Poisson distribution is a normal distribution curve approximately centred at the average degree of oligomerisation. A Schulz-Flory distribution describes a product distribution having a greater molar amount of the small oligomers with a broader range of chain lengths. For short chain oligomers C<12 mainly Schulz-Flory distributions are desired, however, if chain length above C12 are requested Poisson distributed products are often desired. A Gaussian distribution is characterized by the following formula:

$$f ( x \vert μ , {σ}^{2} ) = \frac{1}{σ \sqrt{2 π}} {e}^{- \frac{{( x - μ )}^{2}}{2 {σ}^{2}}}$$

wherein μ is the mean or expectation of the distribution (and also its median and mode), σ is the standard deviation and σ² is the variance.

Linear alpha olefins (LAOs) are valuable commodity chemicals used as precursors in many areas of industry. Annually, more than 3 Mio. tons of alpha olefins are produced globally. In addition, linear alpha-olefins are used in many final products in various applications. For example, the light olefin fractions, 1-butene, 1-hexene and 1-octene, are used as co-monomers in the polymer market, in particular for the production of LLDPE (Linear Low Density Polyethylene) and EPDM (Ethylene Propylene Diene Monomer) rubber. The middle olefin fractions, such as 1-decene, 1-dodecene and 1-tetradecene are used as raw materials for the production of synthetic oils, detergents and shampoos. The heavy olefin fractions can be used as additives for lubricating oils, surfactants, oil field chemicals, waxes and as polymer compatibilisers.

Commonly, commercial LAO plants produce even-numbered alpha-olefins via ethylene oligomerisation. Different thereto Sasol Chemical Industries produces 1-hexene, 1-octene and also smaller quantities of 1-pentene via Fischer-Tropsch-Synthesis from coal. Here, either the Coal-to-Liquid-process (CtL-process) or the Gas-to-Liquid-process (GtL-process) can be used. In the CtL-process, coal reacts at very high temperatures (above 1000° C.) with water vapour and oxygen to form synthesis gas which, after separation of nitrogen oxides and sulphur dioxide, is reacted via heterogeneous catalysis to form hydrocarbons including alpha-olefins and water. In the GtL-process, natural gas is reacted via addition of oxygen and water vapour to form synthesis gas, and the latter is transformed into hydrocarbons in a Fischer-Tropsch-Synthesis. Both processes have the disadvantage that a broad variety of byproducts, such as paraffins and alcohols, are produced. This means that more pure alpha-olefins become accessible only after purification processes (e.g. DE 10022466 A1). Other industrial-scale procedures for the preparation of alpha-olefins are the cracking of paraffins, the dehydrogenation of paraffins and the dehydration of alcohols, decarboxylation of lactones and fatty acids, or chain growth reactions including the oligomerisation of ethylene (e.g. US 20140155666A1). Since ethylene represents an easily available raw material, the first mentioned methods of production play a minor role. The vast majority of alpha-olefins are produced via oligomerisation of ethylene providing exclusively olefins with an even number of C-atoms which have the highest value for commercial applications (e.g. G. J. P. Britovsek et al., Angew. Chem. Int. Ed. 1999, 38, 428-447; S. D. Ittel et al., Chem. Rev. 2000, 100, 1169-1204). Well known industrially used production processes for LAO's, which are based on the oligomerisation of ethylene are the following:
the oligomerisation reaction in the Shell Higher-Olefin-Process (SHOP) using a nickel complex, providing exclusively a Schulz-Flory distribution from the oligomerisation reaction;
the alpha-Sablin-process which uses a zirconia (Zr) compound in conjunction with an aluminium alkyl co-catalyst, providing exclusively a Schulz-Flory distribution from the oligomerisation reaction;
the IDEMITSU's ethylene oligomerisation process (LINEALENE), which make use of a zirconium based catalyst system in combination with triethylaluminium as co-catalyst, providing exclusively a Schulz-Flory distribution from the oligomerisation reaction;
the Chevron-Phillips-Gulf-process (Gulftene-Schulz-Flory distribution) and the INEOS-ethyl-process (Poisson distribution) which rely on the aluminium alkyl mediated oligomerisation of ethylene and the subsequent nickel (Ni)-catalysed displacement of olefins.

The above mentioned processes yield a broad distribution of molecules having different chain lengths. It is in particular difficult to produce certain chain lengths of LAO's, having a carbon chain number beyond 20 carbon atoms (C20) in an economic feasible manner. Due to the constraints of the standard oligomerisation reaction the products have more branching in the higher range alpha olefins. Hence, the distribution is rather inflexible and only a few percent of C20+ material (higher oligomers) can be obtained. All known processes for producing alpha olefins on an industrial scale result either in a Schulz-Flory or in a Gauss or Poisson distribution, respectively, and cannot be controlled to change from one type of distribution to the other.

In addition to the above mentioned processes, there are different processes which selectively produce a single alpha-olefin in high purity. These include the Chevron-Phillips trimerisation process for the production of 1-hexene, the Sasol tri- and tetramerisation process yielding 1-hexene and 1-octene and the Axens/Sabic Alphabutol-process yielding 1-butene.

However, a need remains to find an olefin oligomerisation processes, which can be carried out at mild reaction conditions and with a high yield, also allowing at the same time to vary the distribution of the oligomerised olefins obtained.

A great variety of catalysts for coordinative chain transfer polymerisation (CCTP) have been proposed in the literature so far. CCTP has so far mainly been used to control and modify molecular weights of high molecular weight polymers with molecular weights of above 10000 g/mol. These transition metal based CCTP catalysts are typically used together with co-catalysts which usually act as chain shuttling agent (CSA). Suitable co-catalysts include alkylzinc, alkylaluminium, alkylaluminium halides and alkyl alumoxanes, commonly used in combination with inert, non-coordinating ion forming compounds (activators), Lewis and Brönstedt acids and mixtures thereof. Such prior art processes are for example disclosed in U.S. Pat. No. 5,276,220; G. J. P. Britovsek et al., Angew. Chem. Int. Ed. 2002, 41, 489-491; WO 2003/014046 A1; W. P. Kretschmer et al., Chem. Eur. J. 2006, 12, 8969-8978; S. B. Amin, T. J. Marks, Angew. Chem. 2008, 120, 2034-2054; I. Haas et al., Organometallics 2011, 30, 4854-4861; and A. Valente et al., Chem. Rev. 2013, 113, 3836-3857; S. K. T. Pillai et al., Chem. Eur. J. 2012, 18, 13974-13978; J. Obenauf et al., Eur. J. Inorg. Chem. 2013, 537-544, EP 2671639 A1 (for zirconium), W. P. Kretschmer et al., Dalton Trans., 2010, 39, 6847-6852 (for lanthanides).

One characteristics of CCTP is that the resulting polymer chains are end-capped with the respective main group metal of the co-catalyst and can be further functionalised (M. Bialek, J. Polym. Sci.: Part A: Polym. Chem. 2010, 48, 3209-3214 and W. P. Kretschmer et al., Dalton Trans. 2010, 39, 6847-6852). Nearly all previously reported catalytic systems suffer from ligand transfer from the CCTP catalyst complex onto the CSA and are therefore not stable at high CSA concentrations. However, in order to apply such catalysts systems economically, it is of outmost importance to make high CSA to CCTP ratios possible, since the CSA have to be transformed into the final product (paraffin, olefin, alcohol).

CCTP typically requires the use of a metal complex as catalyst, a co-catalyst and optionally an activator. In the understanding of the present invention, the co-catalyst is a chain shuttling agent (CSA) and may optionally, but not necessarily, be an activator at the same time.

The activator can be for example a compound different from the chain transfer agent that is not functioning as a chain shuttling agent. Such activator is herein solely named “activator” and is not called a “co-catalyst”.

In order to obtain olefins from such processes use of a chain displacement catalyst (CDC) is required. A typical chain displacement catalyst capable of catalysing an olefin exchange reaction (beta-H elimination) is for example Ni(acac)₂, which is reported to give linear alpha-olefins such as disclosed in U.S. Pat. No. 4,918,254, U.S. Pat. No. 6,444,867, U.S. Pat. No. 5,780,697 and U.S. Pat. No. 5,550,303.

All processes known from the prior art use main group metal alkyls in stoichiometric amounts. Hence, the processes known from the prior art are in a need to operate via a two-step process carried out in two different reactors. EP2671639 A1 teaches a novel guanidinato group 4 metal catalyst system, which catalyses the chain growth on aluminium via CCTP.

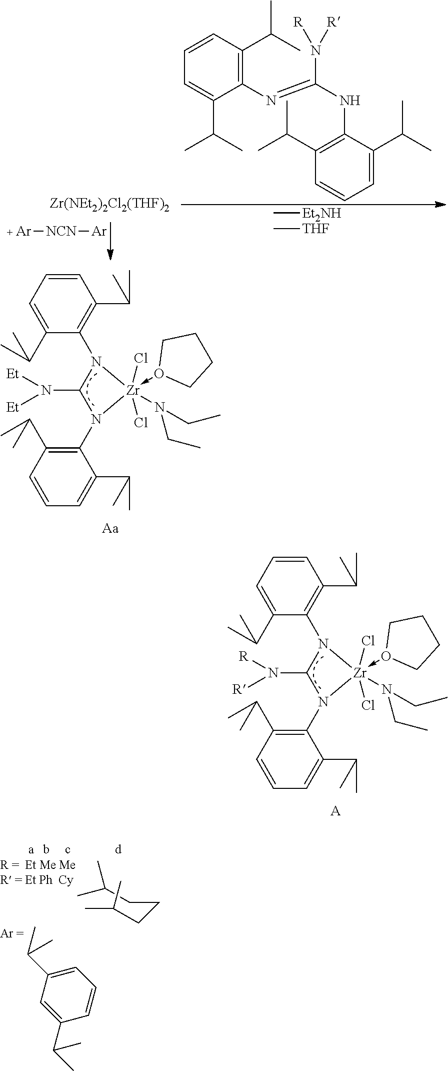

Synthesis of Guadinato Zirconium Complex from EP2671639 A1

The {N′,N″-bis[2,6-di(isopropyl)phenyl]-N,N-diethyl-guanidinato}-(diethylamido)-dichlorido-zirconium(VI); Aa {[Et₂NC(2,6-Prⁱ₂C₆H₃N)₂](Et₂N)ZrCl₂(THF); catalyst was prepared from (Z)-2,3-bis(2,6-diisopropylphenyl)-1,1-diethylguanidine or Bis(2,6-diisopropylphenyl)carbodiimide by reaction with Bis(N,N-diethylamido)-dichlorido-zirconium(IV)-bis(tetrahydrofuran), (Et₂N)₂ZrCl₂(THF)₂, in situ. Unfortunately, applying this in-situ method complex side product formation is increased.

### OBJECT OF THE PRESENT INVENTION

The object of the present invention is to find a highly flexible process which is capable of oligomerising or co-oligomerising alpha-olefins, preferably as desired in either Gaussian, Poisson- or a Schulz-Flory-distribution, at very mild process conditions and with very high catalyst activities over a wide range of CSA amounts. In addition, a process is needed for the in-situ generation of alpha-olefins with the use of known CCTP catalysts systems, which so far have not been stable at high ratios of CCTP to CSA. It is a further object of the present invention to provide via an easy synthesis well-defined catalysts in a high yield.

According to a further embodiment of the present invention following a dual chain shuttling mechanism it is a further object to enhance the chain transfer rate, which results in an increase of the chain transfer (Kₜ) to chain growing (Kₚ) ratio and therefore allows a better control of the produced olefin distribution. It is a further object of the invention to provide additional measures to tune the distribution of the produced olefins thereby also allowing control of the chain length of the produced olefins as well as of the chain length distribution.

### SUMMARY OF THE INVENTION

The present invention is defined by the independent claims. Preferred embodiments are disclosed in the subordinate claims or described hereunder.

The present invention is concerned with a process capable for producing differently distributed oligomerised olefins, including linear olefins, branched olefins, alpha-olefins and/or internal olefins, particular linear olefins at mild conditions, in a flexible manner. In accordance with this invention a process is provided for preparing linear and/or branched oligomerised olefins, particularly linear alpha-olefins including waxes.

The invention makes use of a CCTP catalyst system operating preferably at temperatures between 20-200° C. which comprises a metal organic complex capable of oligomerising or co-oligomerising alpha-olefins as gases or liquids, an activator, at least one chain shuttling agent (CSA), which is capable of transferring the alkyl chain at the catalyst onto the chain shuttling agent, and a chain-displacement-catalyst (CDC) capable of catalysing the beta-H-elimination and if necessary isomerisation to finally obtain olefins (alpha and/or internal olefins) with a controlled chain length distribution.

The oligomerisation according to the present invention can be conducted at high CCTP to CSA and low CCTP to CSA ratios. Surprisingly many CCTP catalyst systems have a higher stability in the presence of a CDC, in particular at a ratio of CCTP/CDC of 1:1 and above, as defined herein.

One advantage of the in situ use of the two catalysts (CCTP, CDC) is that the CSA can be used in catalytic amounts. The obtained olefins can vary in chain length and distribution, which depends on CCTP, CSA(1), CSA(2), CDC, ratios and the process conditions applied. Preferably, the oligomerised olefins obtained are C4 to C80 olefins, most preferably C16 to C30 olefins.

The further embodiment of the present invention following a dual chain shuttling mechanism uses a mixture of a zinc hydrocarbyl compound with a metal alkyl from the groups XII and XIII, preferably trialkylaluminium as chain transfer agents. The zinc hydrocarbyl compound enhances the chain transfer rate, which results in an increase of the chain transfer (Kₜ) to chain growing (Kₚ) ratio to better tune the chain lengths of the produced alpha-olefins. Increasing amounts of zinc hydrocarbyl compounds give shorter chain length and vice versa.

The following improvements can be attributed to the further embodiment of the present invention following dual chain shuttling:
1) CSA(1) and CSA(2) can be used in catalytic amounts.
2) This method allows an improved tuning of the chain lengths of the oligomerised olefin and their distribution.
3) The stability, selectivity, and the activity of a variety of activated CCTP catalysts are enhanced.

The proposed mechanism for the first embodiment is displayed in FIG. 1 and for dual chain shuttling in FIG. 1a, wherein M stands for the CCTP catalyst; CSA(1) and CSA(2) for the chain shuttling agents; diethylzinc as CSA (1) and triethylaluminium as CSA (2) are shown as examples; as CDC (chain displacement catalyst) in FIG. 1a Ni(cyclooctadiene)₂ is shown as example.

The mechanism displayed in FIG. 1a. differs from the mechanism displayed in FIG. 1, in that the oligomeric chain is no longer liberated from the CSA by the CDC but an intermediate CSA(2) cycle is used so that the CSA(1) transfers the oligomeric chain first to the CSA(2) whereupon the CDC liberates the olefin generated from the CSA(2). As in the scheme of FIG. 1 subsequent a fast alkyl exchange with the CSA(2), e.g. triethyl aluminum, transports the oligomeric chain to the CDC, e.g. bis(1,5-cyclooctadiene)nickel(0), which replaces the oligomer by an ethyl group.

It shall be understood that the schemes of FIG. 1 and FIG. 1a display only a proposal for an mechanism to explain how the CSA(s) are regenerated in order to operate with CSA(s) only in catalytic amounts and the present inventors do not wish to be bound to said theory.

The invention is systematically described by the following listing of items:

Item 1: Process for the manufacture of oligomerised olefins by bringing in contact with each other

I Simultaneous Process:
(a) one or more C2 to C8 olefins,
(b) a coordinative chain transfer polymerisation catalyst (CCTP catalyst) comprising one or more organometallic transition metal compounds and one or more ligands,
(c) a chain shuttling agent (CSA) being one or more metal hydrocarbyls selected from the groups II, XII and XIII, or
(c.1) if dual chain shuttling is applied at least two CSA with one being one or more zinc hydrocarbyl compounds (CSA(1)) and the other being one or more XIII metal hydrocarbyl (CSA(2)) preferably aluminium hydrocarbyls, most preferably triethylaluminium,
(d) a chain displacement catalyst (CDC) being one or more members selected from the group consisting of a nickel salt, a cobalt salt, an organo metallic nickel complex and an organo metallic cobalt complex,

to form a growth composition thereby obtaining oligomerised olefins having an oligomerisation degree of 2 to 100.

II Sequential Process:

In the sequential process (d) is brought in contact at a later point with the reaction mixture comprising (a), (b), (c) or (c1), preferably (c), when the oligomerisation has commenced or has come to an end and (b) is at least partially or completely transformed into an inactive reaction product or inactive degradation product.

The simultaneous process I is preferred over sequential process II.

Item 2: The process according to item 1, wherein the growth composition further comprises an activator for the coordinative chain transfer polymerisation catalyst (CCTP catalyst) being an aluminium or boron containing compound comprising at least one hydrocarbyl group.

Item 3: The process according to item 1 or item 2, wherein the olefin is one or more member selected from the group consisting of ethylene, propylene, 1-butene, 1-pentene and 1-hexene, preferably one or more member selected from the group ethylene, propylene or ethylene and propylene.

Item 4: The process according to one or more of the preceding items, wherein the one or more organometallic transition metal compounds comprise one or two transition metals, preferably one transition metal, selected independent from each other from group III, group IV, preferably Ti or Zr, most preferably Zr, group V, group VI, group IX or group X, of the periodic table (according to IUPAC).

Item 5: The process according to one or more of the preceding items, wherein one or two ligands are selected from cyclopentadienyl (preferably 1,3-hydrocarbyl cyclopentadienyl), indenyl, fluorine, diamide ligands, phenoxy-imine-ligand, indolide-imine-ligands, amidinate, guanidinate, amidopyridine, in particular hydrocarbyl-2-pyridyl amine (preferably in combination with one cyclopentadienyl ligand), pyridinimine, and alcoholates each optionally substituted.

Item 6: The process according to one or more of the preceding items, wherein the CCTP catalyst is deactivated during or after the oligomerisation by heating the growth composition, most preferably above 120° C. or by bringing the CCTP catalyst in contact with a catalyst poison, preferably a halogen containing compound, preferably an halogenated aluminium hydrocarbyl.

Item 7: The process according to any one of the preceding items wherein the chain shuttling agent (CSA) is a C1 to C30 hydrocarbyl metal compound, methylalumoxane or both, the metal being aluminium, zinc, magnesium, indium or gallium, preferably trihydrocarbyl aluminium, dihydrocarbyl magnesium or dihydrocarbyl zinc.

Item 8: The process according to any one of the preceding items wherein the chain displacement catalyst (CDC) is selected from nickel halogenides, cobalt halogenides, nickel cyclooctadiene, cobalt cyclooctadiene, nickel acetylactonate, C1 to C30 carboxylic acid salts of nickel and mixtures thereof.

Item 9: The process according to any one of items 2 to 8 wherein the activator is methyl aluminoxan, or a perfluorated aluminate or a boron containing compound or combinations thereof and the boron containing compound preferably comprises one or more members selected from the group consisting of tris(pentafluoro phenyl) borane, tetrakis(pentafluoro phenyl) borate, tris(tetrafluoro phenyl) borane and tetrakis(tetrafluoro phenyl) borate.

Item 10: The process according to any one of the preceding items wherein the molar ratio of the coordinative chain transfer polymerisation catalyst (CCTP catalyst) to the chain shuttling agent (CSA) is 1:>50000 or 1:50 to 1:10000, except for methyl alumoxane as the CSA, wherein the molar amount of CSA refers to all CSA(s) (CSA(1) and CSA(2)) present if more than one CSA is present.

If dual chain shuttling is applied the molar ratio of the coordinative chain transfer polymerisation catalyst (CCTP catalyst) to the chain shuttling agent CSA(1), being a zink alkyl compound, preferably is 1:10 to 1:500. If dual chain shuttling is applied the molar ratio of the coordinative chain transfer polymerisation catalyst (CCTP catalyst) to the chain shuttling agent CSA(2), being an trialkyl aluminium, preferably is 1:50 to 1:500.

Item 11: The process according to any one of the preceding items wherein the molar ratio of the coordinative chain transfer polymerisation catalyst (CCTP catalyst) to the chain displacement catalyst (CDC)
prior or during the oligomerisation is 1:0.5 to 1:50, preferably 1:1 to 1:2, and
after the oligomerisation the concentration of the chain displacement catalyst (CDC) is between 1 to 10000 ppm, preferably 1 to 100 ppm (w/w) relative to the growth composition.

Item 12: The process according to any one of items 2 to 11 wherein the molar ratio of the coordinative chain transfer polymerisation catalyst (CCTP catalyst) to the activator is 1:1 to 1:4, preferably 1:1.05 to 1:2, except for methyl alumoxane.

Item 13: The process according to any one of the preceding items wherein the growth composition further comprises a liquid reaction medium, the liquid reaction medium comprising aromatic hydrocarbons, particularly toluene, linear and/or branched C4 to C20 hydrocarbons and mixtures thereof; cyclic and acyclic hydrocarbons such as cyclohexane, cycloheptane, and/or methylcyclohexane.

Item 14: The process according to any one of the preceding items wherein the reaction is carried out at an ethene or propene or ethene and propene pressure of 0.2 to 60 bar, preferably 1 to 20 bar; most preferably 1 to 10 bar or a 1-butene pressure of 0.2 to 20 bar, preferably 1 to 10 bar.

Item 15: The process according to any one of the preceding items wherein the reaction is carried out at a temperature of 20 to 200° C., preferably of 50 to 100° C.

Item 16: The process according to one or more of the preceding items, wherein the coordinative chain transfer polymerisation catalyst comprises as transition metal Ti, Zr or Hf and one ligand per metal of the following formula

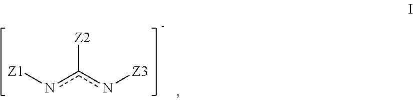

the ligand being bound to the metal, wherein
Z1, Z2 and Z3=are independently hydrocarbon or heteroatom containing hydrocarbon moieties, wherein the heteroatom, if present, for Z1 or Z3 is not directly adjacent to the N-atom and, wherein Z1, Z2 and Z3 independently from each other are optionally linked with one or more of each other.

Item 17: The process according to one or more of the preceding items, wherein the coordinative chain transfer polymerisation catalyst comprises as transition metal Ti, Zr or Hf and one ligand per metal having the following sub-structural formula

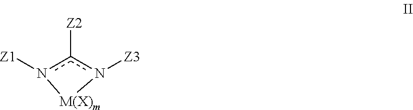

wherein
Z1,Z3=each are independently from each other a di-ortho substituted aromatic moiety,
   each being independently hydrocarbon moieties or heteroatom containing hydrocarbon moieties, wherein the heteroatom, if present, is not directly adjacent to the N-atom,
Z2=is a hydrocarbon moiety or a heteroatom containing hydrocarbon moiety, Z1, Z2 and Z3 independently from each other are optionally linked with one or more of each other, and
M=titanium, zirconium of hafnium.

Item 18: The process of item 17 wherein Z2 is NR1R2 with R1 and R2 independently from each other are C1 to C40 hydrocarbon moieties, optionally comprising one or more heteroatoms.

Item 19: The process according to any one of the preceding items, wherein the coordinative chain transfer polymerisation catalyst (CCTP catalyst) or its active species comprises an M-AIR3 group with M=transition metal and R=C1 to C6 hydrocarbyl.

Item 20: A process for the manufacture of a di-μ-halogen-bridged bis guanidinato tetrahalogen di zirconium compound comprising the following steps:
bringing together
a zirconium amido compound having the following formula

Zr(Hal)₃(NR¹R²)etherate

wherein
Hal is independent from each other halogen, in particular Cl;
R¹, R² being C1 to C40 hydrocarbyl-, optionally comprising one or more heteroatoms, wherein the heteroatom is not adjacent to the N-atom;
the etherate preferably being a di-(C1- to C6-)hydrocarbylether, in particular a di(C1- to C6-)alkylether, a di(C2- or C3-)hydrocarbylether, in particular a di(C2- or C3)alkylether;

and

an carbodiimid-compound having the following formula

(R³)ₓAr—N═C═N—Ar(R⁴)_{y}

wherein
R³, R⁴=independent from each x, y is hydrocarbyl, in particular alkyl, or halogen, wherein R is preferably substituted at the 2 or 6 position of the aryl, and further wherein R is branched at the 2-position
x,y=0 to 3 independent of each other;
Ar=is aryl, optionally substituted, in particular benzene.

in a solvent.

Item 21: The process according to item 20 wherein the di-μ-halogen-bridged bis guanidinato tetrahalogen di zirconium compound is

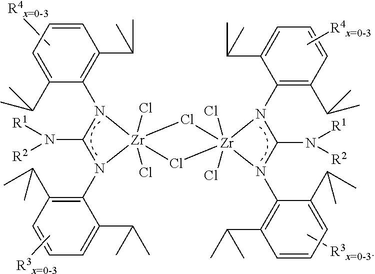

with

R¹, R² being C1 to C40 hydrocarbyl-, optionally comprising one or more heteroatoms, wherein the heteroatom is not adjacent to the N-Atom;
R³, R⁴=independent from each x, y is hydrocarbyl, in particular alkyl, or halogen, wherein R is preferably substituted at the 2 or 6 position of the aryl, and further wherein R is branched at the 2-position;
x=independent from each R³ or R⁴ 0 to 3.

Item 22: The process according to item 20 or 21 wherein the reaction is carried out in a hydrocarbon solvent in particular an aromatic solvent, preferably at temperatures of 30 to 100° C., in particular 40 to 90° C.

Item 23: The process according to one of items 20 to 22 wherein the di-μ-halogen-bridged bis guanidinato tetrahalogen di zirconium compound, preferably as further defined under item 21, is obtained by precipitation, preferably by crystallisation.

Item 24: A process for the manufacture of a zirconium guanidinato alkyl compound comprising the following steps:
bringing together

a di-μ-halogen-bridged bis guanidinato tetrahalogen di zirconium compound, preferably as further defined under item 21,

with a Grignard-reagent, wherein the Grignard-reagent is preferably used in a 2.8 to 3.2 times molar excess relative to the Zr.

Item 25: The process of item 24 wherein independent from each other the di-μ-halogen-bridged bis guanidinato tetrahalogen di zirconium compound, preferably as further defined under item 21, is obtainable by the process of any of items 20 to 23

the Grignard-reagent is alkyl Mg Hal, wherein

Hal is independent from each other halogen, in particular Cl;

Alkyl is C1 to C20 alkyl, in particular methyl or ethyl.

Item 26: The process of item 24 or 25 wherein the zirconium guanidinato alkyl compound is

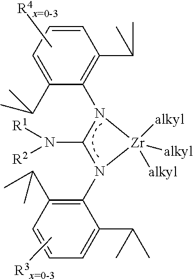

with
R¹, R² being C1 to C40 hydrocarbyl-, optionally comprising one or more heteroatoms, wherein the heteroatom is not adjacent to the N-Atom;
R³, R⁴=independent from each x, y is hydrocarbyl, in particular alkyl, or halogen, wherein R is preferably substituted at the 2 or 6 position of the aryl, and further wherein R is branched at the 2-position;
x=independent from each R³ or R⁴ 0 to 3.

Item 27: The process according to any one of items 24 to 26

wherein the reaction is carried out in a solvent and the solvent is a hydrocarbon, preferably a saturated C4- to C14-hydrocarbon and/or

wherein the zirconium guanidinato alkyl compound is obtained by precipitation, preferably by crystallisation.

Item 28: Use of the compound obtained according to the process of items 24 to 28 in the process of any one of items 1 to 19 as a CCTP catalyst.

Item 29: The process according to one of the items 1-19, wherein the zirconium cyclopentadienyl hydrocarbyl-2-pyridyl amine alkyl compound is

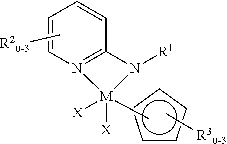

wherein
R1 and R2=independent from each other is hydrocarbyl, in particular alkyl, or halogen, wherein R2 is preferably bound to the 4 and/or 6 position of the aryl, and further wherein R2 is branched at the 2-position;
R3=i is independently from each other zero to three hydrocarbyl, in particular alkyl moieties
and
M=titanium. zirconium of hafnium.
X=independent of each other halogen, preferably Cl; hydrocarbyl, C1 to C40, preferably C1 to C14, in particular methyl and alkylsubstituted cyclopentadiene.

### DETAILED DESCRIPTION OF THE INVENTION

Hereinafter the components of the catalyst system applied and the growth composition are described in detail.

1 CCTP Catalyst and their Ligands

1.1 the Coordinative Chain Transfer Polymerisation (CCTP) Catalyst Comprises One Transition Metal Compounds Selected from (According to IUPAC)
group III, in particular scandium, yttrium, lanthanum, samarium and actinium;
group IV, in particular titanium or zirconium, most preferably zirconium;
group V, in particular vanadium and niobium;
group VI, in particular chromium,
group VIII, in particular iron,
group IX, in particular cobalt;
group X, in particular nickel, palladium and platinum

of the periodic table. Useful ligands, one or two per transition metal are selected from cyclopentadienyl, indenyl, fluorine, diamide ligands, phenoxy-imine-ligand, indolide-imine-ligands, amidinate, guanidinate, amidopyridine, pyridinimine and alcoholate each optionally substituted.

1.1.1 Group IV

Particularly, preferred metals are Ti, Zr or Hf in the +2, +3 or +4 formal oxidation state, preferably in the +4 formal oxidation state.

1.2 A Particularly Preferred Ligand is a Guanidine-Based Metal-Complex Comprising One of the Following Ligands:

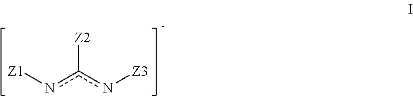

with

Z2=NR1R²,

R1 and R2 are independently from each other hydrocarbon moieties, in particular C1 to C40, preferably C1 to C18, optionally substituted hydrocarbon moieties additionally comprising (not directly adjacent to the N-Atom) one or more nitrogen, oxygen, and/or silicon atom(s), further optionally linked with each other or with Z1 and/or Z3.

Z1 and Z3 independently from each other are:
hydrocarbon moieties, in particular C1 to C40, preferably C3 to C22, most preferably C8 to C18 or more preferably C10 to C22, optionally linked with each other or with Z2, Z1 and Z3 optionally additionally comprising one or more nitrogen, oxygen, and/or silicon atom(s) (not directly adjacent to the N-Atom);
preferably alkyl in particular C1 to C40, preferably C3 to C22, most preferably C8 to C18, or aryl moieties, in particular C6 to C22, most preferably C8 to C18, optionally further substituted by hydrocarbyl groups, in particular C1 to C12, preferably C2 to C6, in particular alkyl, alkenyl or aryl groups, Z1 and Z3 optionally additionally comprising one or more nitrogen, oxygen and/or silicon atom(s) (not directly adjacent to the N-Atom); and
substituted phenyl, in particular tolyl, in particular substituted in the 2 and/or 6 position, mono- or di- or tri-isopropyl phenyl, in particular 2,6-di-isopropyl phenyl, mono- or di- or tri-t-butyl phenyl, in particular 2,6 di-t-butyl phenyl, mono- or di- or tri-(C1 to C4)alkoxy phenyl, in particular 2,6-di-(C1 to C4)alkoxy phenyl, or mono- or di-(C1 to C4)alkylamino phenyl, in particular 2,6-di-(C1 to C4) alkylamino phenyl.

Z1 and Z3 each comprise more carbon atoms than Z2, for example Z1 and Z3 each comprise 8 carbon atoms and more. Most preferably and independent of the above Z1 and Z3 are branched or substituted in one or more of the 2-positions.

1.3 the Metal Complexes Preferably have the Following Structure

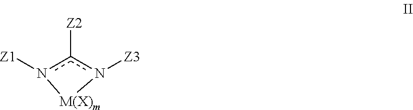

wherein

M=Ti, Zr or Hf, preferably Ti or Zr, more preferably Zr,

X=independent of each m halogen, preferably Cl; hydrocarbyl, in particular C1 to C40, preferably C1 to C4, in particular methyl; hydride; alkoxide; amide, optionally substituted, NR1R2 with R1 and R2 as defined above, preferably NR1R2 is diethylamido, dimethylamido or methylethylamido; tetrahydrofuran; m=1 to 4, with Z1, Z2 and Z3 as defined above.

Most preferably the metal complex has the following structure:

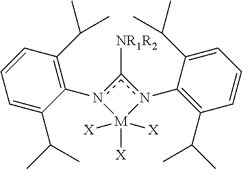

wherein

M=Ti, Zr, preferably Zr

X=halogene, preferably Cl, more preferably hydrocarbyl, in particular methyl, preferably NR1R2 is diethylamido, dimethylamido or methylethylamido

The above mentioned complexes as defined by structures II may also exist as anionic species with an additional cation Q⁺ which for example is selected from the group of R₄N⁺, R₃NH⁺, R₂NH₂⁺, RNH₃⁺, NH₄⁺, R₄P⁺ in which R is an alkyl, aryl, phenyl, hydrogen or halogen.

Examples of the above metal catalysts include
{N′,N″-bis[2,6-di(isopropyl)phenyl]-N,N-dimethyl-guanidinato}metal(IV) chloride,
{N′,N″-bis[2,6-di(isopropyl)phenyl]-N,N-diethyl-guanidinato}metal(IV) chloride,
{N′,N″-bis[2,6-di(isopropyl)phenyl]-N,N-pentamethylene-guanidinato} metal (IV) chloride,
{N′,N″-bis[2,6-di(isopropyl)phenyl]-N-cyclohexyl-N-methyl-guanidinato} metal (IV) chloride,
{N′,N″-bis[2,6-di(isopropyl)phenyl]-N-cyclohexyl-N-methyl-guanidinato} metal (IV) chloride,
[Diethylammonium][N,N′-bis(2,6-diisopropylphenyl)-benzamidinato-tetrachloro]metalat(IV),
[Diethylammonium][N,N′-bis(2,6-diisopropylphenyl)-4-(dimethylamino)benzamidinato-tetrachloro]metalat(IV),
[Diethylammonium][N,N′-bis(2,6-diisopropylphenyl)-4-methoxybenzamidinato-tetrachloro]metalat(IV),
[Diethylammonium][N,N′-bis(2,6-diisopropylphenyl)-4-(2,5-dimethyl-1H-pyrrol-1-yl)benzamidinato-tetrachloro]metalat(IV),
[N,N′-bis(2,6-diisopropylphenyl)-4-(dimethylamino)benzamidinato-diethylamido]trimethylmetal(IV),
[N,N′-bis(2,6-diisopropylphenyl)-4-(2,5-dimethyl-1H-pyrrol-1-yl)benzamidinato-diethylamido]trimethylmetal(IV),
{N′,N″-bis[2,6-di(isopropyl)phenyl]-N,N-dimethyl-guanidinato}trimethylmetal(IV),
{N′,N″-bis[2,6-di(isopropyl)phenyl]-N,N-diethyl-guanidinato}trimethylmetal(IV),
{N′,N″-bis[2,6-di(isopropyl)phenyl]-N,N-pentamethylene-guanidinato}trimethylmetal (IV),
{N′,N″-bis[2,6-di(isopropyl)phenyl]-N-cyclohexyl-N-methyl-guanidinato} trimethylmetal (IV),
{N′,N″-bis[2,6-di(isopropyl)phenyl]-N-cyclohexyl-N-methyl-guanidinato} trimethylmetal (IV) chloride,

preferably with metal=titan or zirconium.

1.3.1 Hydrocarbyl-2-Pyridyl Amine Ligand and Complex

A preferred ligand for metal complexes for the dual chain shuttling is a pyridine amine-based metal-complex comprising one of the following ligands:

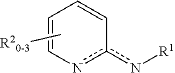

with
R1 is a hydrocarbyl moiety, in particular C1 to C40, preferably C1 to C18, optionally substituted hydrocarbyl moiety additionally comprising one or more nitrogen, oxygen, and/or silicon atom(s)
R2=independent from each other are zero to three hydrocarbyl, in particular alkyl, or halogen moieties, wherein R2 is preferably bound to the 4 or 6 position of the aryl, and further wherein R2 is branched at the 2-position;

The metal complexes preferably have the following structure

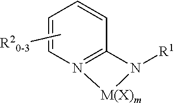

wherein

M=Ti, Zr or Hf, preferably Ti or Zr, more preferably Zr,

X=independent of each m halogen, preferably Cl; hydrocarbyl, C1 to C40, preferably C1 to C14, in particular methyl and alkylsubstituted cyclopentadiene

Most preferably the metal complex has the following structure:

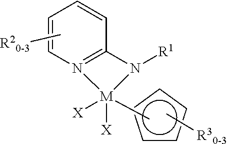

wherein

M=Ti, Zr, preferably Zr

X=halogene, preferably Cl, more preferably hydrocarbyl, in particular methyl, R1, R2 as defined above. R3 is a hydrocarbon moiety, in particular C1 to C40, preferably C1 to C18, optionally substituted hydrocarbon moiety additionally comprising one or more nitrogen, oxygen, and/or silicon atom(s).

The above mentioned complexes may also exist as anionic species with an additional cation Q⁺ which for example is selected from the group of R₄N⁺, R₃NH⁺, R₂NH₂⁺, RNH₃⁺, NH₄⁺, R₄P⁺ in which R is an alkyl, aryl, phenyl, hydrogen or halogen.

Examples of the above metal catalysts include
(1,3-di-tert-butylcyclopenta-1,3-dienyl)-(N-(2,6-diisopropylphenyl)pyridin-2-amidinato)-dimethanidozirconium
(1,3-di-tert-butylcyclopenta-1,3-dienyl)-(6-chloro-N-(2,6-diisopropylphenyl)pyridin-2-amidinato)-dimethanidozirconium(IV)

Alternatively, the metal complex may be formed in situ from suitable transition metal and ligand precursors.

1.3.2: The Transition Metal Precursor May be any Ti, Zr or Hf Complex Capable of Reacting with a Ligand Precursor to Form a Guanidinate Complex or Hydrocarbyl-2-Pyridyl Amine Complex as Described Above In Situ.

Examples of such transition metal precursor (with M=Ti, Zr or Hf) include:
MX₄ where each X may independently halogen {F, Cl, Br, I}, hydride {H}, hydrocarbyl {R, e.g. benzyl}, alkoxide {OR} or amide {NR1R2});
MX₄L₂ where each X may independently halogen {F, Cl, Br, I}, hydride {H}, hydrocarbyl {R, e.g. benzyl}, alkoxide {OR} or amide {NR1R2} with L equals any two electron donor ligand, e.g. ethers such as tetrahydrofuran, or diethytether, acetonitrile, or trihydrocarbylphosphine;
M(acac)₄, where acac=2,4-pentanedionato, 1,1,1,5,5,5-hexafluoro-2,4-pentanedionato or 2,2,6,6-tetramethyl-3,5-heptanedionato;
M(O₂CR)₄, where O₂CR is any carboxylic acid anion, e.g. 2-ethylhexanoate.

The ligand precursor may be any compound capable of reacting with a transition metal precursor to form an amidine or guanidine complex or the cyclopentadienyl and the hydrocarbyl-2-pyridyl amine ligand in situ. Examples of such ligand precursor include:
dihydrocarbylcarbodiimides, such as bis(2,6-diisopropylphenyl)carbodiimide or dicyclohexylcarbodiimide,
diheterohydrocarbylcarbodiimides, such as bis(2-methoxyphenyl)carbodiimide;
amidate or guanidate salts, e.g. lithium 1,3-dihydrocarbylamidate or lithium 1,3-dihydrocarbylguanidate;
guanidines, such as 2,3-bis(2,6-diisopropylphenyl)-1,1-dihydrocarbylguanidine; or
cyclopentadienes or cyclopentadienyl salts such as
1,3-di-tert-butylcyclopenta-1,3-diene
2-pyridine amines or 6-pyridine amines such as
N-(2,6-diisopropylphenyl)pyridine-2-amine.

1.4. The Metal Complexes Become a Catalyst for CCTP when Combined at Least with a Co-Catalyst.

The co-catalyst, without being bound to the theory, acts as a chain shuttling agent and may optionally act in addition as an activator for the complex in order that the complex becomes the (active) catalyst.

2.0 Activator

The activator may comprise a boron containing compound such as a borate. More preferably the activator comprises pentafluorophenyl boranes and pentafluorophenyl borates. Illustrative examples of boron compounds which may be used as activator in the preparation of catalysts of this invention are tri-substituted (alkyl) ammonium salts such as

trimethylammonium tetraphenylborate, triethylammonium tetraphenylborate, tripropylammonium tetraphenylborate, tri(n-butyl)ammonium tetraphenylborate, tri(t-butyl)ammonium tetraphenylborate, N,N-dimethylanilinium tetraphenylborate, N,N-diethylanilinium tetraphenylborate, N,N-dimethyl-2,4,6-trimethylanilinium tetraphenylborate, trimethylammonium tetrakis(pentafluorophenyl) borate, triethylammonium tetrakis(pentafluorophenyl) borate, tripropylammonium tetrakis(pentafluorophenyl) borate, tri(n-butyl)ammonium tetrakis(pentafluorophenyl) borate, tri(sec-butyl)ammonium tetrakis(pentafluorophenyl) borate, dioctadecylmethylammonium tetrakis(pentafluorophenyl)borate, dioctadecylmethylammonium tetrakis(3,5-bis(trifluoromethyl)-phenyl)borate, N,N-dimethylanilinium tetrakis(pentafluorophenyl) borate, N,N-dimethylanilinium n-butyltris(pentafluorophenyl) borate, N,N-dimethylanilinium benzyltris(pentafluorophenyl) borate,

N,N-dimethylanilinium tetrakis(4-(t-butyldiimethylsilyl)-2,3,5,6-tetrafluorophenyl) borate, N,N-dimethylanilinium tetrakis(4-(triisopropylsilyl)-2,3,5,6-tetrafluorophenyl) borate, N,N-dimethylanilinium pentafluorophenoxytris(pentafluorophenyl) borate, N,N-diethylanilinium tetrakis(pentafluorophenyl) borate, N,N-dimethyl-2,4,6-trimethylanilinium tetrakis(pentafluorophenyl) borate, trimethylammonium tetrakis(2,3,4,6-tetrafluorophenyl) borate, triethylammonium tetrakis(2,3,4,6-tetrafluorophenyl) borate, tripropylammonium tetrakis(2,3,4,6-tetrafluorophenyl) borate, tri(n-butyl)ammonium tetrakis(2,3,4,6-tetrafluorophenyl) borate, dimethyl(t-butyl) ammonium tetrakis(2,3,4,6-tetrafluorophenyl) borate, N,N-dimethylanilinium tetrakis(2,3,4,6-tetrafluorophenyl) borate, N,N-diethylanilinium tetrakis(2,3,4,6-tetrafluorophenyl) borate, and N,N-dimethyl-2,4,6-trimethylanilinium tetrakis(2,3,4,6-tetrafluorophenyl) borate; dialkyl ammonium salts such as: di-(i-propyl)ammonium tetrakis(pentafluorophenyl) borate, and dicyclohexylammonium tetrakis(pentafluorophenyl) borate; tri-substituted phosphonium salts such as: triphenyiphosphonium tetrakis(pentafluorophenyl) borate, tri(o-tolyl)phosphonium tetrakis(pentafluorophenyl) borate, and tri(2,6-dimethylphenyl)phosphonium tetrakis(pentafluorophenyl) borate;

di-substituted oxonium salts such as: diphenyloxonium tetrakis(pentafluorophenyl) borate, di(o-tolyl)oxonium tetrakis(pentafluorophenyl) borate, and di(2,6-dimethylphenyl)oxonium tetrakis(pentafluorophenyl) borate;

di-substituted sulfonium salts such as: diphenylsulfonium tetrakis(pentafluorophenyl) borate, di(o-tolyl)sulfonium tetrakis(pentafluorophenyl) borate, and bis(2,6-dimethylphenyl)sulfonium tetrakis(pentafluorophenyl) borate.

The activator may alternatively comprise an aluminium containing compound such as an aluminate. More preferably the activator comprises an tetrakisalkyloxy-aluminate or tetrakisaryloxy-aluminate, in particular tetrakis-C1 to C6-alkyloxy-aluminate or tetrakisaryloxy-aluminate, the alky or aryl being —CF₃ substituted, such as [Al(OC(Ph)(CF₃)₂)₄]⁻ or [Al(OC(CF₃)₃)₄]⁻.

3.0 Chain Shuttling Agent CSA (=Co-Catalyst)

The CSA a chain shuttling agent (CSA) being one or more metal alkyls selected from the group II, XII and XIII from the periodic table. The CSA preferably is a C1 to C30 hydrocarbyl metal compound, methylaluminoxane or both, the metal being aluminium, zinc, magnesium, indium or gallium, preferably trihydrocarbyl aluminium, dihydrocarbyl magnesium or dihydrocarbyl zinc, preferrably zink dialkyl.

According to one embodiment of the invention it is preferred to use a mixture in particular a mixture of tri C1- to C3-alkyl aluminium and di-C1- to C3-alkyl zinc.

If dual chain shuttling is applied the CSAs are preferably Zn alkyl compounds (CSA(1)) and the other being one or more XIII metal alkyl (CSA(2)) preferably aluminium alkyls, most preferably triethylaluminium, Most preferably the CSA or CSAs (CSA (1), CSA (2)) (co-catalysts) are selected from:
tri hydrocarbyl aluminium, wherein the hydrocarbyl is for example methyl, ethyl, propyl, isopropyl, n-butyl, isobutyl, pentyl, neopentyl or isopentyl or a mixtures thereof, preferably tri(methyl and/or ethyl) aluminium,
di-hydrocarbyl zinc, wherein the hydrocarbyl is for example methyl, ethyl, propyl, isopropyl, n-butyl, isobutyl, pentyl, neopentyl or isopentyl or a mixtures thereof, preferably di(methyl and/or ethyl) zinc, or any other zinc compound that forms under reaction conditions zinc dihydrocarbyl compounds.
a mixture of tri hydrocarbyl aluminium and di-hydrocarbyl zinc reagents as described above,
oligomeric or polymeric hydrocarbyl alumoxanes, preferably oligomeric or polymeric methyl alumoxanes (including modified methylalumoxane, modified by reaction of methylalumoxane with triisobutyl aluminium or isobutylalumoxane),

and for single CSA activation, not dual CSA:
hydrocarbyl aluminium halogenides such as dialkyl aluminium halogenides, alkyl aluminium dihalogenides, with alkyl preferably being C1 to C3-alkly, hydrocarbyl aluminium sesqui halogenides, preferably. methyl aluminium sesqui halogenides,
di-hydrocarbyl magnesium, wherein the hydrocarbyl is for example methyl, ethyl, propyl, isopropyl, n-butyl, isobutyl, pentyl, neopentyl or isopentyl or a mixtures thereof, preferably di(methyl and/or ethyl and/or butyl) magnesium; tri-hydrocarbyl indium, wherein the hydrocarbyl is for example methyl, ethyl, propyl, isopropyl, n-butyl, isobutyl, pentyl, neopentyl or isopentyl or a mixtures thereof, preferably tri(methyl and/or ethyl and/or butyl) indium; tri-hydrocarbyl gallium, wherein the hydrocarbyl is for example methyl, ethyl, propyl, isopropyl, n-butyl, isobutyl, pentyl, neopentyl or isopentyl or a mixtures thereof, preferably tri(methyl and/or ethyl and/or butyl) gallium or mixture thereof.

The most preferred CSA (acting also as co-catalyst) for use in forming the (active) catalysts is triethylaluminium or a mixture of triethylaluminium comprising minor portions of diethylaluminiumhydrid (such as below 10 wt. %).

Regarding the dual chain shuttling using a Zn/Al combination, the inventors assume without limiting the invention thereto that the zinc hydrocarbyl compound (CSA(1)) transfers the chains from and to the CCTP catalyst and that zinc hydrocarbyl compounds increase the chain transfer rate. This results in an increase of the chain transfer (Kₜ) to chain growing (Kₚ) ratio. The aluminum hydrocarbyl CSA(2) is believed to shuttle the chains from CSA(1) to the chain displacement catalyst.

5.0 Activated CCTP Catalyst

The active CCTP catalysts are rendered catalytically active by combination of a CCTP catalyst (see 1.0 CCTP catalyst and ligands) with a) an activating co-catalyst (CSA) (on its own) or b) by a combination of a co-catalyst (CSA) with an activator as listed under 2.0 activator.

In addition to above mentioned co-catalysts an activator can be used or is preferably to be used when the co-catalyst on its own is not activating. If the respective cocatalyst is selected from the trialkyl aluminium compounds use of activator is preferable. Suitable activators are referenced above.

The foregoing co-catalysts and activating techniques have been previously taught with respect to different metal complexes in the following references: EP 277003, U.S. Pat. No. 5,153,157, U.S. Pat. No. 5,064,802, EP 468651 and EP 520732, the teachings of which are hereby incorporated by reference.

The molar ratio of catalyst (CCTP catalyst) to co-catalyst (CSA) with reference to the [metal catalyst] to [CSA] atomic ratio preferably is from 1:1 to 1:10000000, more preferably 1:100 to 1:100000 and most preferably 1:1000 to 1:40000.

6.0 Chain Displacement Catalyst (CDC)

The chain displacement catalyst is a nickel or cobalt compound. Typical compounds are nickel and cobalt compounds with one or more of the following substituent: halides, carbonyls, acetylacetonato, cyclooocta-1,5-diene, cyclopentadienyl, C1- to C12-octanoates, tri(C1- to C12-hydrocarbyl)-phosphines.

Most preferred are bis(cyclooctadienyDnickel(0) and nickel(II) acetylacetonate.

7.0 Carrier

A support, especially silica, alumina, magnesium chloride, or a polymer (especially poly(tetrafluoroethylene or a polyolefin) may also be applied. The support is preferably used in an amount to provide a weight ratio of catalyst (based on metal):support from 1:100000 to 1:10, more preferably from 1:50000 to 1:20, and most preferably from 1:10000 to 1:30.

8.0 Solvent

Suitable solvents for oligomerisation are preferably inert liquids. Suitable solvents include aliphatic and aromatic hydrocarbons, particularly C4 to C20 hydrocarbons or olefins, linear and/or branched, and mixtures thereof (including monomers subject to oligomerisation, especially the previously mentioned addition polymerisable monomers and produced oligomerised olefins); cyclic and alcyclic hydrocarbons such as cyclohexane, cycloheptane, methylcyclohexane, methylcycloheptane, and mixtures thereof; isooctanes, aromatic and hydrocarbyl-substituted aromatic compounds such as benzene, toluene, and xylene. Mixtures of the foregoing are also suitable. Most preferred is toluene.

9.0 Olefins

In accordance with this invention C1- to C8-olefins, particularly alpha olefins, especially ethylene or ethylene and propylene or propylene are converted to oligomeric mono-unsaturated hydrocarbons, in short herein called oligomerised olefins.

10.0 Process (Conditions Applicable to all Modes of Operation)

The Process for the manufacture of oligomerised olefins comprises

bringing in contact with each other at least
(a) the one or more olefins as defined above,
(b) the coordinative chain transfer polymerisation catalyst (CCTP) as defined above,
(c) the chain shuttling agent (CSA) as defined above, or
(c.1) if dual chain shuttling is applied at least two CSAs one being one or more Zn hydrocarbyl compounds (CSA(1)), preferably dihydrocarbyl zinc, the other being one or more XIII metal hydrocarbyls (CSA(2)) preferably aluminium alkyls, most preferably triethylaluminium,
(a), (b) and (c)/(c.1) form the obligatory components of the growth composition, preferably in one reactor.

The growth composition contains further before or during the oligomerisation the chain displacement catalyst (CDC) according to one embodiment (simultaneous process)

The products obtained are the oligomerised olefins described herein below.

The order of bringing the components together is not of particular relevance. Nevertheless typically a solvent is provided first and the solvent is saturated with the olefin.

Suspension, solution, slurry, gas phase, solid state powder oligomerisation or other process condition may be applied as desired.

In general, the oligomerisation may be accomplished at temperatures from 20 to 200° C., preferably 50 to 100° C., most preferably 60-90° C., and pressures from 1 to 100 bar, preferably 1 to 30 bar. In general, shorter olefins can be produced if the reaction temperature is increased and pressure is decreased.

The distribution can be shifted from Schulz-Flory to Poisson or Gaussian via the applied CCTP catalyst system, the CSA, the activator and displacement catalyst. The distribution can additionally be tuned by the catalyst:CSA:CDC ratio, and furthermore for the dual chain shuttling reaction mode by the CSA(1):CSA(2) ratio. The distribution can also be altered via temperature and applied pressure. The produced olefins can be purified via mechanical or thermal purification processes. In general filtration and distillation can be applied for purification purposes.

In the single CSA process for obtaining an oligomerised olefin by applying CCTP and subsequently the CDC, the olefin is obtained in a Poisson or Gaussian distribution, wherein the molar ratio of the coordinative chain transfer polymerisation catalyst (CCTP catalyst) to the chain shuttling agent (CSA) is 1:>10000, preferably 1:>100000. The chain length can be tuned by the amount of olefin oligomerised.

It was found that higher concentrations or an increase of the (partial) pressures of the C2- to C8-olefin results in a higher oligomerisation degrees. Higher means above 4 bar.

It was further found that a higher reaction temperature results in a lower oligomerisation degree.

The process according to the invention can be carried out in three different modes:
a) via a simultaneous process where a CCTP catalyst, a CSA and a CDC (and optionally an activator) are present at the same time, e.g. from the start; or
b) via a sequential process where at first a CCTP catalyst, and a CSA (and optionally activator) are present but no CDC and at a later stage the CCTP is deactivated and the CDC is added; or
c) via a simultaneous process where CCTP catalyst, CSA(1), CSA(2) and CDC (and optionally activator) are present at the same time, e.g. from the start.

10.1 Simultaneous Process

If in the regular reaction mode CCTP and CDC both are present in the reaction composition, chain growth and chain displacement take place at the same time and high ratios of CSA to CCTP and of CDC to CCTP result in short chain lengths with a Schulz-Flory distribution.

However, if in the single CSA reaction mode low CSA concentrations (CCTP/CSA 1:<1000, in particular 1:100 to 1:500) and low CDC concentrations (CCTP/CDC 1:1 to 1:2) are applied, mainly a Gauss or Poisson distribution is obtained. In general the product distribution can either be tuned by the applied type of CCTP, CSA and CDC and by the ratio of CCTP/CSA/CDC or by the applied reaction conditions, mainly pressure and temperature. Increasing ethylene pressure results in higher molecular weight olefins and broader distribution, while increasing temperature yields more short chain olefins with a more narrow distribution.

However, the biggest influence on the type of distribution of chain lengths obtained has the applied CCTP catalyst system. For instance a CCTP catalyst with a higher transfer to propagation rate (Kₜ≥Kₚ, Scheme 2), e.g. guanidinato zirconium complexes, yields at equivalent reaction conditions mainly a Schulz-Flory distribution of the oligomerised olefins, while a CCTP catalyst with Kₜ≤Kₚ, e.g. guanidinato titanium catalysts, gives mainly Poisson or Gaussian distributions.

When applying the simultaneous reaction mode CCTP and CDC (as well as CSA(1) and CSA(2)) are present during most of the reaction time typically resulting in oligomerised olefins, wherein more that 80 wt % of olefins are obtained in a Schulz-Flory distribution, wherein the molar ratio of the coordinative chain transfer polymerisation catalyst (CCTP catalyst) to the chain shuttling agent (CSA) is 1:<10000, preferably 1:<1000; and the molar ratio of the coordinative chain transfer polymerisation catalyst (CCTP catalyst) to the chain displacement catalyst (CDC) is 1:>2, preferably 1:5 to 1:10.

According to a different way of conducting the simultaneous reaction mode the oligomerised olefins are being obtained in a mainly Poisson or Gaussian distribution, wherein the molar ratio of the coordinative chain transfer polymerisation catalyst (CCTP catalyst) to the chain shuttling agent (GSA) is 1:<10000, preferably 1:<1000; and the molar ratio of the coordinative chain transfer polymerisation catalyst (CCTP catalyst) to the chain displacement catalyst (CDC) is 1:<10, preferably 1:<4.

The olefin is further preferably obtained in a Schulz-Flory distribution, if the molar ratio of the coordinative chain transfer polymerisation catalyst (CCTP catalyst) to the chain shuttling agent (CSA) is 1:>1000, preferably 1:>10000; and the molar ratio of the coordinative chain transfer polymerisation catalyst (CCTP catalyst) to the chain displacement catalyst (CDC) is 1:>10, preferably 1:10 to 1:20.

If the process is carried out with a C2 or C3 or C2 and C3 olefin and a pressure of lower than 4 bar, preferably lower than 2 bar, the oligomerised olefin is being obtained predominately in a Schulz-Flory distribution.

If the dual CSA system is applied and a Schulz Flory distribution of the oligomerised olefins is desired, the Zr/CSA molar ratio preferably is between 1:300 and 1:500 and the Zr/CDA molar ratio is between 1:10 to 1:20 and the CSA(1)/CSA(2) molar ratio is greater than 4:1.

If the dual CSA system is applied and a Poisson or Gaussian distribution of the oligomerised olefins is desired, the Zr/CSA molar ratio preferably is between 1:300 and 1:500 and the Zr/CDA molar ratio is between 1:10 to 1:20 and the CSA(1)/CSA(2) molar ratio is smaller than 4:1.

The process can be performed in one single reactor without any intermediate steps or transfers.

10.2 Sequential Process

According to one embodiment of the invention comprising a sequential reaction the CDC is subsequently brought in contact with the reaction composition comprising the inactivated CCTP catalyst, the CSA and the oligomerised olefin, the reaction composition not comprising the CDC, wherein the CCTP catalyst is inactivated by heating the reaction composition, most preferably above 120° C. or adding catalysts poisons, the catalyst poisons being preferably selected from the group consisting of halogenated metal alkyls alkali and earth alkali salts, the catalysts poisons being selected in a manner that the CCTP catalyst is inactivated but not the CSA and not the CDC to be added.

For the sequential reaction mode it is preferred to use a molar ratio of the coordinative chain transfer polymerisation catalyst (CCTP catalyst) to the chain displacement catalyst (CDC) of 1:0.05 to 1:100, preferably 1:1 to 1:2. Optionally the molar ratio of the coordinative chain transfer polymerisation catalyst (CCTP catalyst) to the chain shuttling agent (CSA) is preferably 1:>50000. For the sequential reaction mode the CDC catalyst is preferably added at a temperature above 120° C.

The sequential reaction mode results in a Schulz-Flory distribution, of the oligomerised olefins at low molar ratios of olefin to CSA. Otherwise the sequential reaction mode results in a predominantly Poisson or Gaussian distribution, in particular if the C2 to C3 pressure is greater than 2 bar. In other words in case of sub-sequent addition of CDC and at CSA conversion above 20% increasing amounts of CSA give shorter chain length with a mainly Poisson or Gaussian distribution, below 20% conversion the process will result in a product with a mainly Schulz-Flory distribution.

11.0 Product

Desirably the oligomerisation is conducted by contacting the monomer(s) and catalyst composition under conditions to produce an oligomer or a polymer having molecular weight (MW [g/mol]) from 56 to 1000000, preferably 56 to 10000, most preferably 84 to 1000.

In particular it may be wanted that high molecular oligomers (>1000 g/mol) are produced. For determination of the molecular weight distribution gel permeation chromatography (GPC) or mass spectroscopy may be used. For olefins having molecular weight below 1000 standard gas chromatography can be applied.

GPC-samples were prepared by dissolving the polymer (0.05 wt.-%, conc.=1 mg/mL) in the mobile phase solvent in an external oven and were run without filtration. The molecular weight was referenced to polyethylene (Mw=520 to 3200000 gmol⁻¹) and polystyrene (Mw=580 to 2800000 gmol⁻¹).

The distribution of the chain lengths of the olefins obtained can be influenced as follows:

Simultaneous Process with Use of a Single CSA

| | | | | | Dominant |
|---|---|---|---|---|---|
| | | | | | distribution |
| CCTP | CSA | CDC | CCTP:CSA | CCTP:CDC | of products |
| IV (Zr) | TEAL | Ni | 1:<1000 | 1:<4 | Gauss or Poisson |
| IV (Zr) | TEAL | Ni | 1:<1000 | 1:5-1:10 | Schulz-Flory |
| IV (Zr) | TEAL | Ni | 1:>10000 | 1:10-1:20 | Schulz-Flory |
| I (Ti) | TEAL | Ni | 1:>10000 | 1:<20 | Gauss or Poisson |

Simultaneous Process with Use of Dual CSA

| | | | | | | Dominant distribution of |
|---|---|---|---|---|---|---|
| CCTP | CSA(1) | CSA(2) | CDC | CCTP:CSA | CSA(1):CSA(2) | products |
| IV (Zr) | DEZn | TEAL | Ni | 1:<1000 | <4:1 | Gauss or Poisson |
| IV (Zr) | DEZn | TEAL | Ni | 1:<1000 | >4:1 | Schulz-Flory |

Sequential Process with Use of a Single CSA

| | | | CSA | Dominant distribution |
|---|---|---|---|---|
| CCTP | CSA | CDC | conversion | of products |
| IV (Zr) | TEAL | Ni | >20% | Gauss or Poisson |
| IV (Zr) | TEAL | Ni | <20% | Schulz-Flory |
| I (Ti) | TEAL | Ni | 10-100% | Gauss or Poisson |

11. Synthesis of Preferred CCTP Catalysts

In the preparation of the CCTP catalyst highly selective synthesis routes are preferred as intermediate compounds and by products also exhibit oligomerisation activity often yielding undesired molecular weight olefins. The most preferred catalysts according to this invention are complexes IV which are obtained with high selectivity by reacting Zr(NEt₂)Cl₃(Et₂O) with Ar—NCN—Ar in a first step to obtain III and reacting III in a second step with 6 moles of methyl magnesium chloride in hexane.

The process for the manufacture of a preferred zirconium guanidinato alkyl compound comprises the following steps:
bringing together a di-μ-halogen-bridged bis guanidinato tetrahalogen di zirconium compound
with a Grignard-Reagent, wherein the Grignard-Reagent is preferably used in a 2.8 to 3.2 times molar excess relative to the Zr.

The Di-μ-halogen-bridged bis guanidinato tetrahalogen di zirconium compound is obtainable for example by the following process:
bringing together in a solvent:
a zirconium amido compound having the following formula

Zr(Hal)₃(NR¹R²)Etherate

wherein
Hal is independent from each other Halogen, in particular Cl;
R¹, R² being C1 to C40 hydrocarbyl-, optionally comprising one or more heteroatoms, wherein the heteroatom is not adjacent to the N-Atom;
the etherate preferably being a di-(C1- to C6-)hydrocarbylether, in particular a di(C1- to C6-)alkylether, a di-(C2- or C3-)hydrocarbylether, in particular a Di(C2- or C3)alkylether;

and

an carbodiimid-compound having the following formula

(R³)ₓ(Ar—N═C═N—Ar(R⁴)_{y}

wherein
R³, R⁴=independent from each x, y is hydrocarbyl, in particular alkyl, or halogen, wherein R is preferably substituted at the 2 or 6 position of the aryl, and further wherein R is branched at the 2-position;
   x,y=independent from each R³ or R⁴ 0 to 3;
   Ar=is Aryl, in particular Benzene.

A preferred Grignard-Reagent is Alkyl Mg Hal, wherein Hal is independent from each other Halogen, in particular Cl, and Alkyl is C1 to C20 alkyl, in particular Methyl or Ethlyl.

The Di-μ-halogen-bridged bis guanidinato tetrahalogen di zirconium compound preferably is

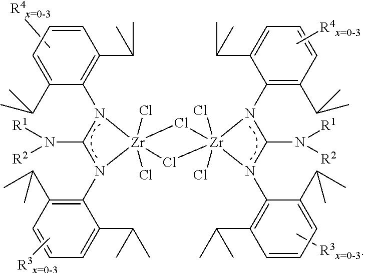

with

x=0 to 3 independent from each R³ or R⁴

R¹, R², R³, R⁴ as defined in claim 18.

A preferred zirconium guanidinato alkyl compound is

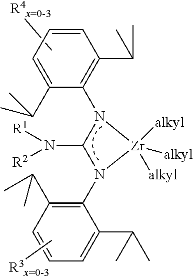

with
R¹, R² being C1 to C40 hydrocarbyl-, optionally comprising one or more heteroatoms, wherein the heteroatom is not adjacent to the N-Atom;
R³, R⁴=independent from each x, y is hydrocarbyl, in particular alkyl, or halogen, wherein R is preferably substituted at the 2 or 6 position of the aryl, and further wherein R is branched at the 2-position;
x=independent from each R³ or R⁴ 0 to 3.

The compound obtained according to the above process can be used as a CCTP catalyst.

The reaction scheme may be outlined as follows:

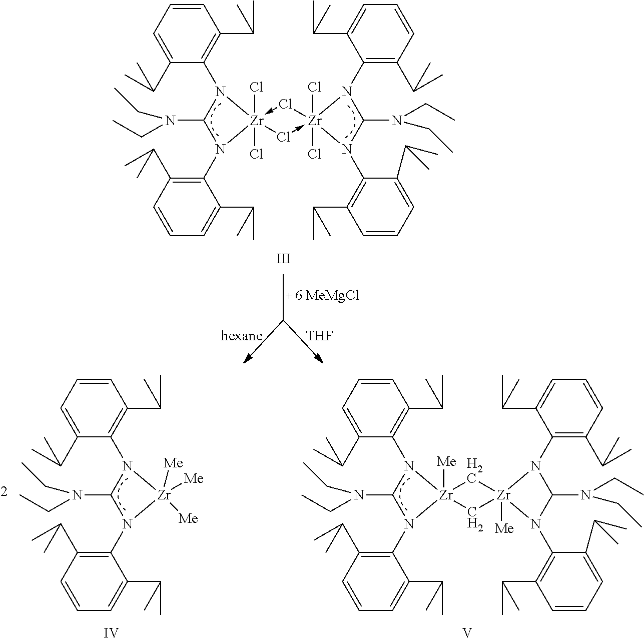

The same reaction in THF yields the methylene bridged dimer di-μ-methylenebis[2,3-bis(2,6-diisopropylphenyl)-1,1-diethylguanidinato]-dimethanido-dizirconium(IV), V μ-CH₂-{[Et₂NC(2,6-Prⁱ₂C₆H₃N)₂]ZrMe₂}₂ instead, which is much less active and less selective. Reacting I with 3 equivalents of Methyl Grignard reagent did not yield the expected trimethyl analog but the less preferred {N′,N″-bis[2,6-di(isopropyl)phenyl]-N,N-dialkyl-guanidinato}-(diethylamido)-dimethanido-zirconium(VI) complexes, II {[RR′NC(2,6-Prⁱ₂C₆H₃N)₂](Et₂N)ZrMe₂, instead.

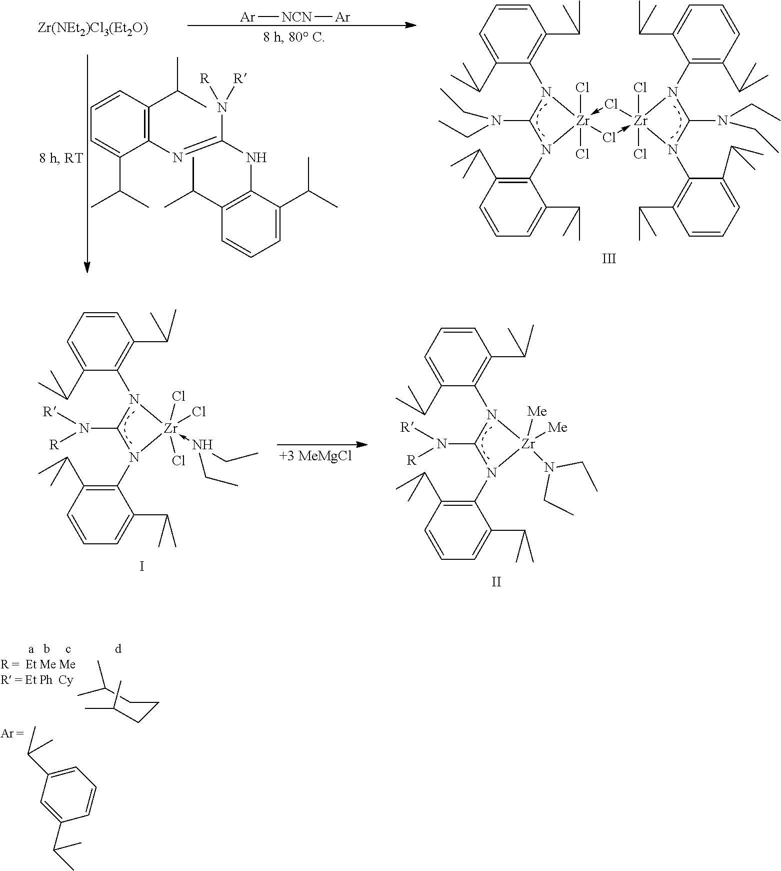

The new well-defined catalyst (structure IV) can therefore improve the earlier described CCTP process by reducing the high molecular weight polymer side products by simultaneously enhancing the catalyst activity.

### FIGURES

The following is depicted in the attached figures:

FIG. 1: Reaction scheme illustrating the assumed mechanism of the tandem catalyst oligomerisation process of ethylene via single chain shuttling

FIG. 1a: Reaction scheme illustrating the assumed mechanism of dual chain shuttling via two CSAs

FIG. 2: Molecular structure of III in the formula as displayed above.

FIG. 3: Molecular structure of IV as described in the formula as displayed above.

FIG. 4: 1H NMR spectrum (C2Cl₄D₂, 120° C.) of oligomers obtained with GuaTiMe₃ (I) precatalyst in absence (entry 1, table 1, above) and presence of Ni(COD)₂ (entry 5, table 2, below). In the absence of a CDC no olefins are observed. With Ni(COD)₂ only alpha-olefins were observed.

FIG. 5: 1H NMR spectrum (C₂Cl₄D2, 120° C.) of oligomers obtained with GuaZrMe₃ (III) precatalyst in absence (entry 2, below, table 1) and presence of Ni(COD)₂ (entry 9, above, table 2). In absence of a CDC no olefins are observed.

FIG. 6: 1H NMR spectrum (C₂Cl₄D₂, 120° C.) of oligomers obtained with GuaZrMe₃ (III) precatalyst in presence of Ni(COD)₂ (entry 9, below, table 2), Ni(acac)₂ (entry 6, middle, table 2) and Ni(stea)₂ (entry 7, above, table 2). From top to the bottom the ratio between terminal to internal olefins increases.

FIG. 7: Oligomerised olefin distribution at different ethylene pressures; 8 μmol GuaZrMe₃, 16 μmol Dimethylaniliniumborat, 32 μmol Ni(COD)₂, 8000 μmol TEAL, T=60° C.

FIG. 8: Oligomerised olefin distribution at different temperatures; 8 μmol GuaZrMe3, 16 μmol Dimethylaniliniumborat, 32 μmol Ni(COD)₂, 8000 μmol TEAL, p=2 bar.

FIG. 9 Oligomerised olefin distribution at different temperatures; 4 μmol GuaZrMe3, 4 μmol Trioctyammonium borate, 8 μmol Ni(COD)₂, 40000 μmol TEAL, p=2 bar, 22 g ethylene.

FIG. 9a Oligomerised olefin distribution using the yttrium complexes of Example S6; 10 μmol yttrium pre-catalyst, 10.0 μmol [R₂N(CH₃)H]⁺[B(C₆F₅)₄]⁻ (R═C₁₆H₃₃—C₁₈H₃₇), 2 μmol Ni(COD)₂, 500 μmol TEAL, p=5 bar, T=80° C.

FIG. 10: ¹H NMR spectrum (C₂Cl₄D₂, 120° C.) of oligomers obtained with Cp“ApZrMe₂ (II) precatalyst and in the presence of 2 μmol Ni(COD)₂ (Table 8, entry 6).

FIG. 11: ¹H NMR spectrum (C₂Cl₄D₂, 120° C.) of oligomers obtained with Cp” Ap^{Cl}ZrMe₂ (II) precatalyst and in the presence of 24 μmol Ni(COD)₂ (Table 8, entry 13).

FIG. 12: Influence of DEZn content on the oligomerised olefin obtained with Cp″ApZrMe₂ (I) precatalyst in presence of Ni(COD)₂ (Table 8, entry 1, Table 8, entries 3-7).

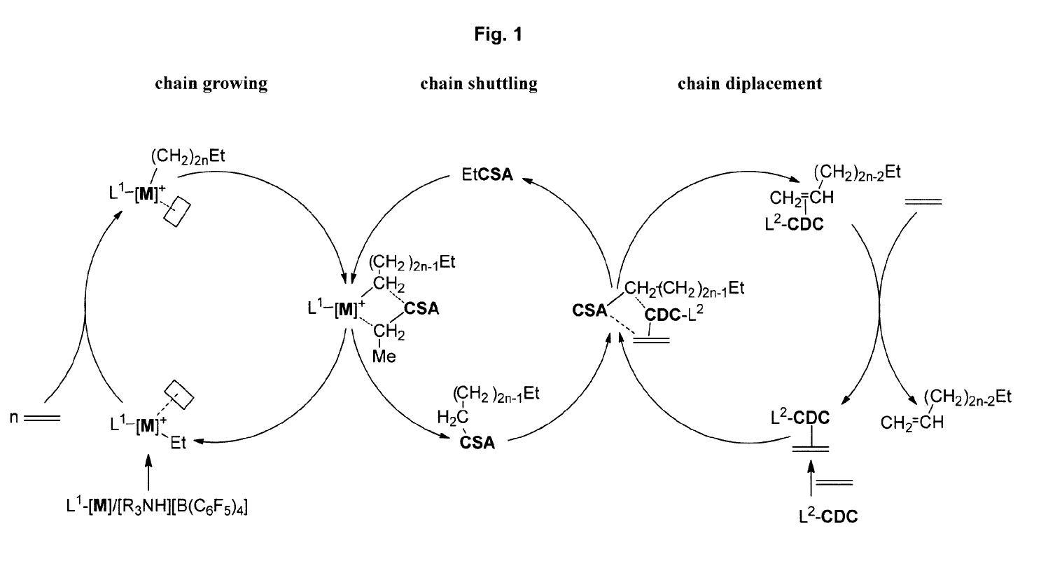

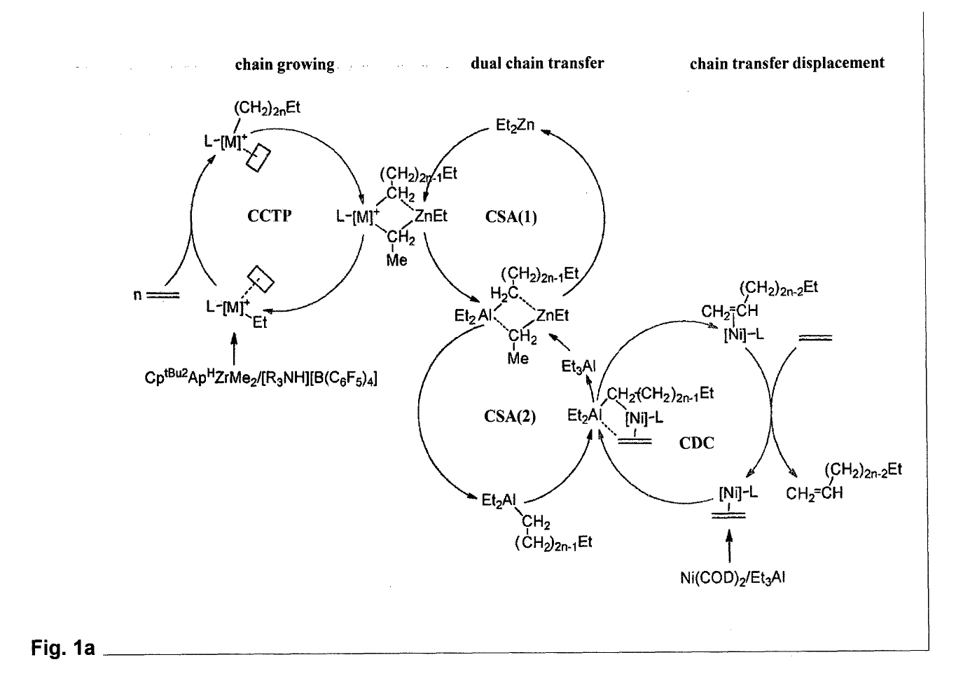

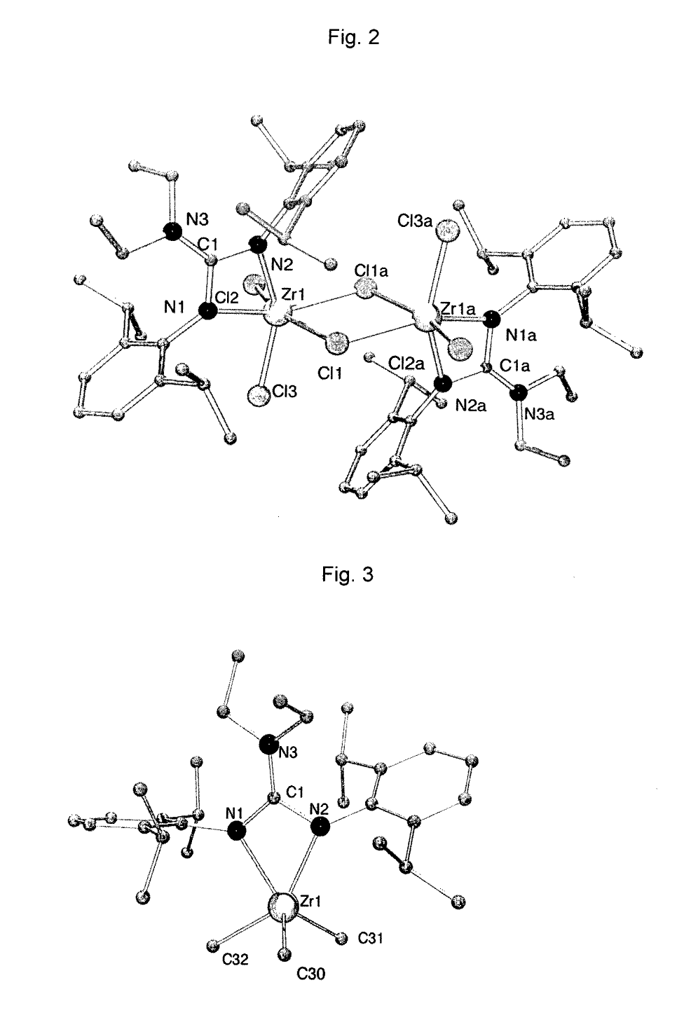

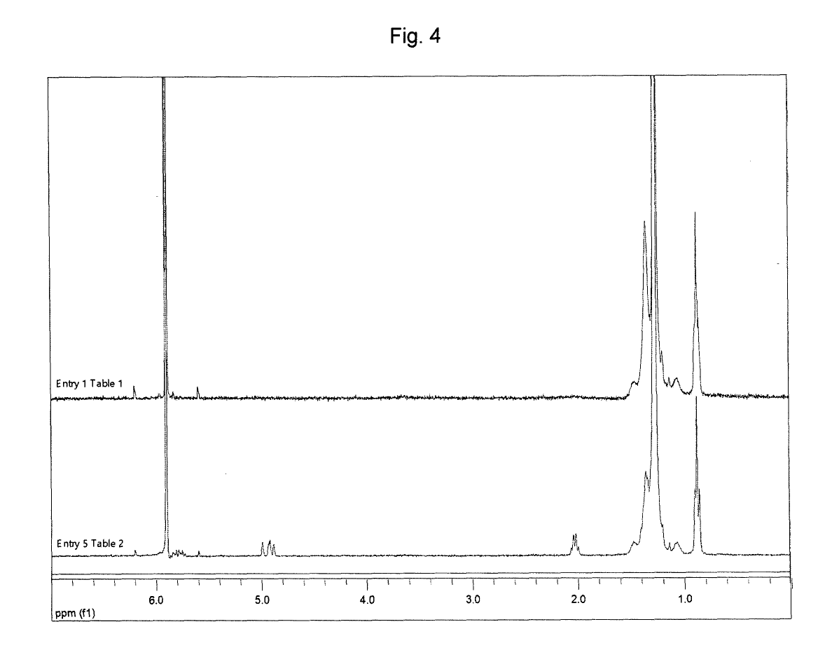

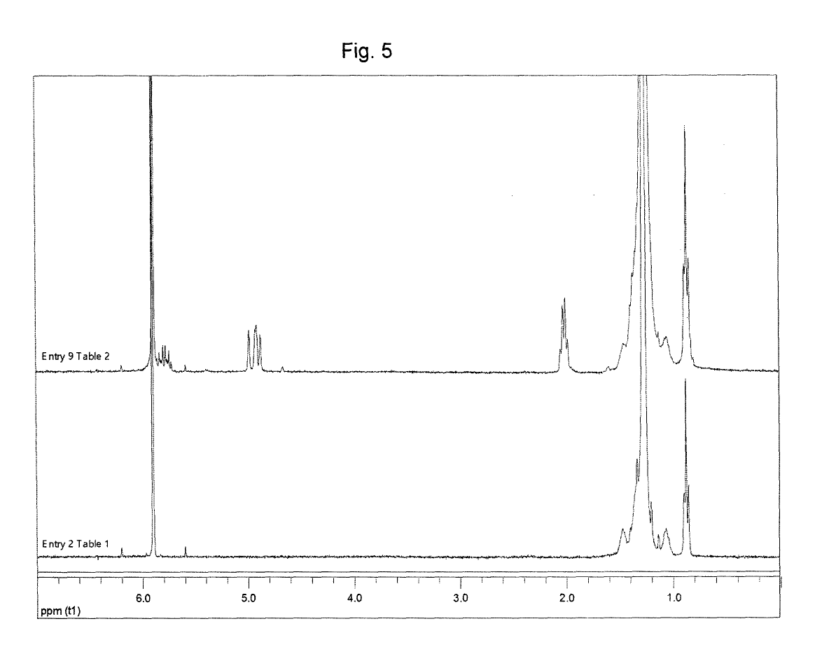

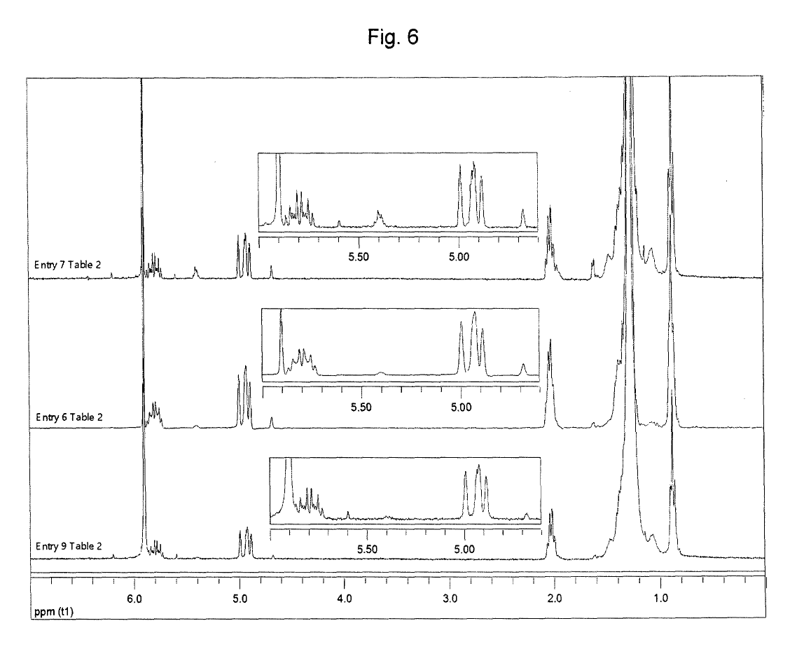

### EXPERIMENTAL SECTION

The following abbreviations were used:
Me—Methyl (CH₃)
Et—Ethyl (CH₃CH₂)
TEAL—Triethyl aluminium (Et₃Al)
GuaH—2,3-bis(2,6-diisopropylphenyl)-1,1-diethylguanidine
i-Pr—iso-Propyl (Me₂CH)
i-Bu—iso-Butyl (Me₂CHCH₂)
Cy—Cyclohexyl (C₆H₁₁)
TMA—Trimethylaluminium (Me₃Al)
TEA—Triethylaluminium (Et₃Al)
DEAC—Diethylaluminiumchloride [(Et₂AlCl)₂]
Pipi—cis-2,6-Dimethylpiperidin-1-yl [cis-2,6-Me₂C₅H₈N]
COD—Cyclooctadiene (C₈H₁₂)
acac—Acetylacetonate (CH₃COCHCOCH₃⁻)
stea—Stearate (O₂C(CH₂)₁₆CH₃⁻)
Ni(acac)₂—Nickel(II) acetylacetonate (Ni(C₅H₇O₂)₂)
Ni(COD)₂—Bis(1,5-cyclooctadiene)nickel(0) (Ni(C₈H₁₂)₂)
Ni(stea)₂—Nickel(II) stearate (Ni(O₂C(CH₂)₁₆CH₃)₂)
DEZn—Diethyl zinc (Et₂Zn)
ApH N-(2,6-diisopropylphenyl)pyridin-2-amine [2-(NC₅H₄)(2,6-PrⁱC₆H₃)NH]
Ap^{Cl}H—6-Chloro-N-(2,6-diisopropylphenyl)pyridin-2-amine [2-(6-ClNC₅H₃)(2,6-PrⁱC₆H₃)NH]
Cp″H—1,3-di-tert-butylcyclopenta-1,3-diene (1,3-Bu^{t}C₅H₄)

All ratios herein are molar ratios except when specifically mentioned otherwise.

General: All manipulations of air- or moisture-sensitive compounds were carried out under N₂ using glove-box, standard Schlenk, or vacuum-line techniques. Solvents and reagents were purified by distillation from LiAlH₄, potassium, Na/K alloy, or sodium ketyl of benzophenone under nitrogen immediately before use. Toluene (Aldrich, anhydrous, 99.8%) was passed over columns of Al₂O₃ (Fisher Scientific), BASF R³-11 supported Cu oxygen scavenger, and molecular sieves (Aldrich, 4 Å). Ethylene and Propylene (AGA polymer grade) were passed over BASF R3-11 supported Cu oxygen scavenger and molecular sieves (Aldrich, 4 Å). NMR spectra were recorded on a Varian Inova 400 (¹H: 400 MHz, ¹³C: 100.5 MHz) or Varian Inova 300 (1H: 300 MHz, 13C: 75.4 MHz) spectrometer. The ¹H and ¹³C NMR spectra, measured at 26° C., were referenced internally using the residual solvent resonances, and the chemical shifts (δ) reported in ppm. High temperature NMR measurements of polymer samples were carried out in deutero tetrachloroethane at 120° C.

Gel permeation chromatography (GPC) analysis was carried out on a PL-GPC 220 (Agilent, Polymer Laboratories) high temperature chromatographic unit equipped with LS, DP and RI detectors and three linear mixed bed columns (Olexis, 13-micron particle size) at 150° C. using 1,2,4-trichlorobenzene as the mobile phase. The samples were prepared by dissolving the polymer (0.05 wt.-%, conc.=1 mg/mL) in the mobile phase solvent in an external oven and were run without filtration. The molecular weight was referenced to polyethylene (Mw=520-3200000 gmol⁻¹) and polystyrene (Mw=580-2800000 gmol⁻¹) standards.

The reported values are the average of at least two independent determinations. GC analysis was performed with an Agilent 6850 gas chromatograph and/or Agilent 7890A GC with an inert MSD 5975C with Triple Axis Detector. Both GC's are equipped with an Agilent 19095J-323E capillary column (HP-5; 5% phenyl methyl siloxane; 30 m; film 1.5 μm, diameter 0.53 mm) and a flame ionization detector.

N,N-Dimethylanilinium (tetrapentafluorophenyl) borate ([PhNMe₂H][B(C₆F₅)₄]), Nickel(II) stearate, Bis(1,5-cyclooctadiene)nickel(0), Nickel(II) pentanedionate (anhydrous; 95%), Titanium(IV)chloride and Zirconium(IV)chloride are commercially available from abcr GmbH & Co. KG. Triethyl aluminium (SASOL Germany GmbH) and Bis(2,6-diisopropylphenyl)carbodiimide (TCI Deutschland GmbH) were used as received. The ligand precursor 2,3-bis(2,6-diisopropylphenyl)-1,1-diethylguanidine (G. Jin, C. Jones, P. C. Junk, K.-A. Lippert, R. P. Rose, A. Stasch, New J. Chem, 2008, 33, 64-75) and the metal precursors diethylaminotrichloridozirconium(IV) etherate (E. V. Avtomonov, K. A. Rufanov, Z. Naturforsch. 1999, 54 b, 1563-1567) and 2,3-Bis(2,6-diisopropylphenyl)-1,1-diethylguanidinato trimethanido titanium(IV) (GuaTiMe₃, I, J. Obenauf, W. P. Kretschmer, R. Kempe, Eur. J. Inorg. Chem. 2014, 1446-1453) were prepared according to published procedures.

### Comparative Example 1: Synthesis of {2,3-bis[2,6-di(isopropyl)phenyl]-1,1-diethyl-guanidinato}-(diethylamido)-dichlorido-zirconium(VI) (Aa; Mixtures Of Isomers)

Method A:

Bis(diethylamido)-dichlorido-zirconium(IV)-bis(tetrahydrofurane) (0.036 g, 80 □mol) and Bis(2,6-diisopropylphenyl) carbodiimide (0.029 g, 80 μmol) were subsequently added to a Schlenk flask filled with 10 mL of toluene and stirred at RT. After 24 h the mixture was filtered and diluted with toluene to reach 40 mL. This solution was used in oligomerisation without further purification.

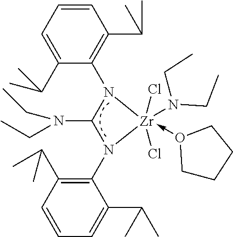

Alternative Method B can be used: Bis(diethylamido)-dichlorido-zirconium(IV)bis(tetrahydrofurane) (0.036 g, 80 μmol) and (Z)-2,3-bis(2,6-diisopropylphenyl)-1,1-diethylguanidine (0.035 g, 80 μmol) were subsequently added to a Schlenk flask filled with 10 mL of toluene and stirred at RT. After 24 h the mixture was filtered and diluted with toluene to reach 40 mL. This solution was used in oligomerisation without further purification.

### Comparative Example 2

General description of ethylene oligomerisation experiments for Runs 1-6

The catalytic ethylene oligomerisation reactions were performed in a 250 mL glass autoclave (Buechi) in semi-batch mode (ethylene was added by replenishing flow to keep the pressure constant). The reactor was ethylene flow controlled and equipped with separated toluene, catalyst and co-catalyst injection systems. During a oligomerisation run the pressure and the reactor temperature were kept constant while the ethylene flow was monitored continuously. In a typical semi-batch experiment, the autoclave was evacuated and heated for 1 h at 80° C. prior to use. The reactor was then brought to desired temperature, stirred at 1000 rpm and charged with 150 mL of toluene. After pressurizing with ethylene to reach 2 bar total pressure the autoclave was equilibrated for 10 min. Successive TEAL co-catalyst solution, activator (perfluorophenylborate) and 1 mL of a zirconium pre-catalyst stock solution in toluene was injected, to start the reaction.

After the desired reaction time the reactor was vented and the residual aluminium alkyls were destroyed by addition of 50 mL of ethanol. Polymeric product was collected, stirred for 30 min in acidified ethanol and rinsed with ethanol and acetone on a glass frit. The polymer was initially dried on air and subsequently in vacuum at 80° C. Oligomeric product was collected by washing the toluene solution with water and removing the solvent under reduced pressure. The oily product was analyzed by GC-MS.

TABLE 1
Ethylene oligomerisation with Zr pre-catalyst Aa, TEAL
co-catalyst and perfluorophenylborate activator.^{a}

| | | | t | m_{product.} | Activity | Mₙ | |
|---|---|---|---|---|---|---|---|
| Entry | Precat | Al/Zr | [min] | [g] | [kg_{PE}mol_{cat}⁻¹h⁻¹bar⁻¹] | [kgmol⁻¹] | M_{w}/Mₙ |
| 1 | Aa | 250 | 15 | 1.68 | 1680 | 1300 | 1.9 |
| 2 | Aa | 500 | 15 | 1.70 | 1700 | 1140 | 1.5 |
| 3 | Aa | 750 | 15 | 3.54 | 3540 | 1030 | 1.5 |
| 4 | Aa | 1000 | 15 | 6.48 | 6480 | 1290 | 1.3 |
| 5 | Aa | 10000 | 30 | 6.50 | 3270 | 350 | 1.3 |
| 6 | Aa | 10000 | 60 | 21.00 | 5250 | 370 | 1.5 |

^{a}Pre-catalyst: 2.0 μmol; ammonium borate: 2.2 μmol [R₂N(CH₃)H]⁺[B(C₆F₅)₄]⁻ (R = C₁₆H₃₃—C₁₈H₃₇), Zr/B = 1/1.1; toluene: 150 mL; T = 50° C., p = 2 bar.

### Example 1: Synthesis of 2,3-Bis(2,6-diisopropylphenyl)-1,1-diethylguanidinato diethylamino trichiorido zirconium(IV) (Ia)

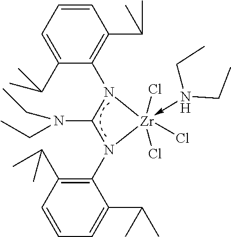

2,3-Bis(2,6-diisopropylphenyl)-1,1-diethylguanidine (2.55 g, 5.85 mmol) and diethylamido-trichloridozirconium(IV) etherate (2.01 g, 5.85 mmol) were dissolved in toluene (100 mL) and stirred overnight. Diethylamine (0.86 mg, 11.16 mmol) was added to the filtered reaction solution and the mixture was stirred for one hour. After filtration and concentration of the reaction solution, colourless crystals were obtained at −30° C. ¹H NMR (300 MHz, C₆D₆): δ=0.15 (t, 6H, CH₃), 0.67 (t, 6H, CH₃), 1.26 (d, 12H, CH₃), 1.59 (d, 12H, CH₃), 2.49 (br s, 4H, CH₂), 2.77 (q, 4H, CH₂), 3.60 (s, 1H, NH), 3.91 (m, 4H, CH), 7.13 (d, 6H, CHₐᵣₒₘ) ppm.

### Example 2: Synthesis of 2,3-bis(2,6-diisopropylphenyl)-C-(cis-2,6-dimethylpiperidyl)guanidinato diethylamido trichlorido zirconium(IV) (Id)

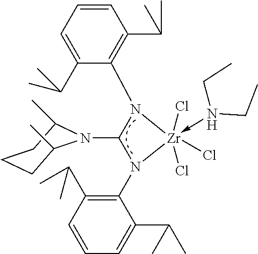

2,3-bis(2,6-diisopropylphenyl)-C-(cis-2,6-dimethylpiperidyl)guanidine (11.2 g, 23.5 mmol) and diethylamido-trichloridozirconium(IV) etherate (8.1 g, 23.5 mmol) were dissolved in toluene (300 mL) and stirred overnight. The reaction solution is filtered, diethylamine (3.0 mL, 28.7 mmol) is added and stirred for one hour. After filtration and concentration of the reaction solution, colourless crystals could be obtained at −30° C. ¹H NMR (300 MHz, C₆D₆): δ=0.73 (d, 6H, CH₃); 0.75 (t, 6H, CH₃); 1.29-1.72 (m, 6H, CH₂); 1.04 (d, 6H, CH₃); 1.40 (d, 6H, CH₃); 1.47 (d, 6H, CH₃); 1.71 (d, 6H, CH₃); 2.54 (q, 4H, CH₂); 3.23 (s, 1H, NH); 3.99 (sept, 4H, CH); 3.60-3.74 (m, 2H, CH); 6.93-7.19 (m, 6H, CHₐᵣₒₘ) ppm.

### Example 3: Synthesis of 2,3-Bis(2,6-diisopropylphenyl)-1,1-diethylguanidinato diethylamido-dimethanido zirconium(IV) (IId)

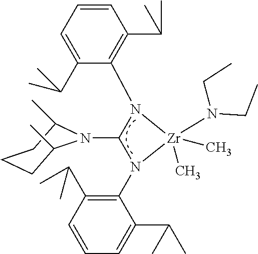

To a suspension of bis(2,6-diisopropylphenyl)-C-(cis-2,6-dimethylpiperidyl)guanidinato-diethylamido-trichlorido-zirconium (2.5 g, 3.4 mmol) in ether (50 mL) methylmagnesium chloride (3 M in THF, 4.9 mL, 14.7 mmol) was added dropwise at −78° C. The mixture was warmed to room temperature and stirred overnight. Storage of the concentrated filtrate at −30° C. led to colourless crystals. Yield 1.9 g (85%). ¹H NMR (300 MHz, C₆D₆): δ=0.53 (s, 6H, CH₃); 0.74 (d, 6H, CH₃); 0.90 (t, 6H, CH₃); 0.80-1.46 (m, 6H, CH₂); 1.09 (d, 6H, CH₃); 1.23 (d, 6H, CH₃); 1.29 (d, 6H, CH₃); 1.37 (d, 6H, CH₃); 3.24 (q, 4H, CH₂); 3.63 3.99 (sept, 4H, CH); 3.88-3.98 (m, 2H, CH); 7.04-7.14 (m, 6H, CHₐᵣₒₘ) ppm.

### Example 4

General Description of Ethylene Oligomerisation Experiments for Runs 7-15

The catalytic ethylene oligomerisation reactions were performed in a 250 mL glass autoclave (Buechi) in semi-batch mode (ethylene was added by replenishing flow to keep the pressure constant). The reactor was ethylene flow controlled and equipped with separated toluene, catalyst and co-catalyst injection systems. During a oligomerisation run the pressure and the reactor temperature were kept constant while the ethylene flow was monitored continuously.

In a typical semi-batch experiment, the autoclave was evacuated and heated for 1 h at 80° C. prior to use. The reactor was then brought to desired temperature, stirred at 1000 rpm and charged with 150 mL of toluene. After pressurizing with ethylene to reach 2 bar total pressure the autoclave was equilibrated for 10 min. Successive TEAL co-catalyst solution, activator (perfluorophenylborate) and 1 mL of a zirconium pre-catalyst stock solution in toluene was injected, to start the reaction. After the desired reaction time the reactor was vented and the residual aluminium alkyls were destroyed by addition of 50 mL of ethanol. Polymeric product was collected, stirred for 30 min in acidified ethanol and rinsed with ethanol and acetone on a glass frit. The polymer was initially dried on air and subsequently in vacuum at 80° C. Oligomeric product was collected by washing the toluene solution with water and removing the solvent under reduced pressure. The oily product was analyzed by GC-MS.

TABLE 2
Ethylene oligomerisation with Zr pre-catalysts example 1-3,
TEAL co-catalyst and perfluorophenylborate activator.^{a}

| | | | t | m_{roduct.} | Activity | Mₙ | |
|---|---|---|---|---|---|---|---|
| Entry | Precat | Al/Zr | [min] | [g] | [kg_{PE}mol_{cat}⁻¹h⁻¹bar⁻¹] | [kgmol⁻¹] | M_{w}/Mₙ |
| 7 | Ia | 500 | 15 | 0.63 | 1150 | 650 | 1.14 |
| 8^{b} | Ia | 5000 | 15 | 1.13 | 1080 | liquid^{d} | — |
| 9 | Id | 500 | 15 | 1.45 | 2900 | 1181 | 1.5 |
| 10 | Id | 1000 | 15 | 4.58 | 9160 | 860 | 1.5 |
| 11^{c} | Id | 1000 | 15 | 10.87 | 21800 | 2590 | 2.5 |
| 12^{c,f} | Id | 72000 | 60 | 68.2 | 14200 | 650 | 1.6 |
| 13^{b,f} | Id | 79000 | 15 | 28.1 | 14080 | liquid^{d} | — |
| 14^{b,e} | Id | 75000 | 15 | 28.5 | 17070 | 280 | 1.6 |
| 15 | IId | 500 | 15 | 3.7 | 7400 | 12450 | 1.9 |

^{a}Precatalyst: 1.0 μmol; ammonium borate: 1.1 μmol [R₂N(CH₃)H]⁺[B(C₆F₅)₄]⁻ (R = C₁₆H₃₃—C₁₈H₃₇), Zr/B = 1/1.1; toluene: 150 mL; T = 50° C., p = 2 bar; t = 15 min.
^{b}Precatalyst: 2.0 μmol.
^{c}anilinium borate: 1.1 mmol [PhN(CH₃)₂H]⁺[B(C₆F₅)₄]⁻.
^{d}oligomeric products.
^{e}3 bar ethylene.
^{f}4 bar ethylene.

### Example 5: Synthesis of Di-μ-chlorido-bis[2,3-bis(2,6-diisopropylphenyl)-1,1-diethylguanidinato]-tetrachlorido-dizirconium(IV) (III)

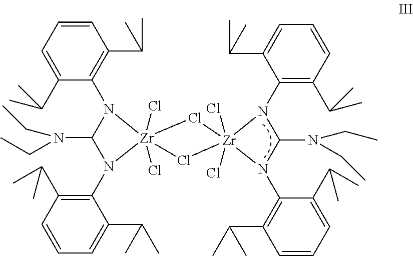

Diethylamido-trichloridozirconium(IV) etherate (0.68 g, 2.0 mmol) and Bis(2,6-diisopropylphenyl) carbodiimide (0.55 g, 1.5 mmol were dissolved in toluene (100 mL) and stirred overnight at 60° C. After filtration and concentration of the reaction solution, colourless crystals were obtained at −30° C. ¹H NMR (300 MHz, C₆D₆): δ=0.20 (t, 6H, CH₃), 1.18 (d, 12H, CH₃), 1.50 (d, 12H, CH₃), 2.59 (q, 4H, CH₂), 3.55 (m, 4H, CH), 7.06 (d, 6H, CHₐᵣₒₘ) ppm. ¹³C NMR (75.4 MHz, C₆D₆): δ=20.5 (CH₃), 25.3 (CH₃), 37.8 (CH), 48.7 (CH₂), 121.1 (Carom), 123.6 (Carom), 125.6 (Carom) ppm.

### Example 6 Synthesis of 2,3-Bis(2,6-diisopropylphenyl)-1,1-diethylguanidinato trimethanido zirconium(IV) (IV)

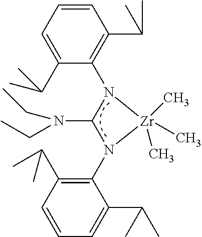

To a suspension of dimeric μ²-Chlorido-[2,3-bis(2,6-diisopropylphenyl)-1,1-diethylguanidinato]trichloridozirconium(IV) (1176 mg, 0.93 mmol) in hexane (50 mL) methylmagnesium chloride (1.9 mL, 5.58 mmol) was added dropwise at −78° C. The mixture was warmed to room temperature and stirred overnight. Storage of the concentrated filtrate at −30° C. led to colourless crystals. Yield 903 mg (85%). ¹H NMR (300 MHz, C₆D₆): δ=0.26 [t, J=7.1 Hz, 6H, N(CH₂CH₃)₂]; 0.86 [s, 9H, Zr(CH₃)_{3]; 1.24} [d, J=6.9 Hz, 12H, CH(CH₃)₂]; 1.36 [d, J=7.1 Hz, 12H, CH(CH₃)₂]; 2.74 [q, J=7.1 Hz, 4H, N(CH₂CH₃)₂]; 3.62 [sept, J=6.8 Hz, 4H, CH(CH₃)₂]; 7.09 (s, 6H, ArH) ppm. ¹³C NMR (75.4 MHz, C₆D₆): δ=11.3 [N(CH₂CH₃)₂]; 24.0 [CH(CH₃)₂]; 25.9 [CH(CH₃)₂]; 28.5 [CH(CH₃)₂]; 40.8 [N(CH₂CH₃)₂]; 51.4 [Zr(CH₃)_{3]; 124.3, 125.5, 142.7, 143.3} (ArC); 169.5 (NCN) ppm.

### Example 7: Synthesis of Di-μ-methylene-bis[2,3-bis(2,6-diisopropylphenyl)-1,1-diethylguanidinato]-dimethanido-dizirconium(IV) (V)

To a suspension of Di-μ-chlorido-bis[2,3-bis(2,6-diisopropylphenyl)-1,1-diethylguanidinato]-tetrachlorido-dizirconium(IV) (1.92 g, 1.52 mmol) in THF (30 mL) methylmagnesium chloride (3.05 mL, 9.12 mmol) was added dropwise at −78° C. The mixture was warmed to room temperature and stirred overnight. Solvent was removed under reduced pressure and the residue extracted twice with hexane (2×20 mL). Storage of the concentrated filtrate at −30° C. led to light yellow crystals. Yield 1.68 g (88%). ¹H NMR (300 MHz, C6D6): δ=0.21-0.27 (m, 6H, N(CH₂CH₃)₂); 0.58 (s, 3H, Zr(CH₃)₃); 1.26 (d, 6H, J=6.8 Hz, CH(CH₃)₂); 1.31 (d, 6H, J=6.8 Hz, CH(CH₃)₂); 1.39 (d, 6H, J=6.7 Hz, CH(CH₃)₂); 1.48 (d, 6H, J=6.7 Hz, CH(CH₃)₂); 2.76 (m, 4H, N(CH₂CH₃)₂); 3.62 (sept., 2H, J=6.8 Hz, CH(CH₃)₂); 3.83 (sept., 2H, J=6.8 Hz, CH(CH₃)₂); 5.25 (s, 2H, Zr(CH₂)Zr); 7.05 (m, 6H, ArH) ppm.

### Example 8

General Description of Ethylene Oligomerisation Experiments for Runs 16-21

The catalytic ethylene oligomerisation reactions were performed in a 250 mL glass autoclave (Buechi) in semi-batch mode (ethylene was added by replenishing flow to keep the pressure constant). The reactor was ethylene flow controlled and equipped with, separated toluene, catalyst and co-catalyst injection systems. During a oligomerisation run the pressure and the reactor temperature were kept constant while the ethylene flow was monitored continuously. In a typical semi-batch experiment, the autoclave was evacuated and heated for 1 h at 80° C. prior to use. The reactor was then brought to desired temperature, stirred at 1000 rpm and charged with 150 mL of toluene. After pressurizing with ethylene to reach 2 bar total pressure the autoclave was equilibrated for 10 min. Successive TEAL co-catalyst solution, activator (perfluorophenylborate) and 1 mL of a 0.001 M zirconium pre-catalyst stock solution in toluene was injected, to start the reaction. After the desired reaction time the reactor was vented and the residual aluminium alkyls were destroyed by addition of 50 mL of ethanol. Polymeric product was collected, stirred for 30 min in acidified ethanol and rinsed with ethanol and acetone on a glass frit. The polymer was initially dried on air and subsequently in vacuum at 80° C. Oligomeric product was collected by washing the toluene solution with water and removing the solvent under reduced pressure. The oily product was analyzed by GC-MS.

TABLE 3
Ethylene oligomerisation with Zr pre-catalyst III and IV,
TEAL co-catalyst and perfluorophenylborate activator.^{a}

| | | | t | m_{product.} | Activity | Mₙ | |
|---|---|---|---|---|---|---|---|
| Entry | Precat | Al/Zr | [min] | [g] | [kg_{PE}mol_{cat}⁻¹h⁻¹bar⁻¹] | [kgmol⁻¹] | M_{w}/Mₙ |
| 16^{b} | III | 2000 | 15 | 1.00 | 500 | 560 | 1.2 |
| 17^{b} | III | 1000 | 15 | 1.40 | 700 | 780 | 1.5 |
| 18^{b} | III | 500 | 15 | 3.24 | 1620 | 990 | 1.5 |
| 19 | IV | 2000 | 15 | 12.73 | 25480 | 3000 | 1.5 |
| 20^{c,e} | IV | 72000 | 22 | 28.13 | 25598 | liquid^{d} | — |
| 21 | IV | 1000 | 15 | 13.38 | 26800 | 2480 | 1.9 |

^{a}Precatalyst: 1.0 μmol; ammonium borate: 1.1 μmol [R₂N(CH₃)H]⁺[B(C₆F₅)₄]⁻ (R = C₁₆H₃₃—C₁₈H₃₇), Zr/B = 1/1.1; toluene: 150 mL; T = 50° C., p = 2 bar; t = 15 min.
^{b}Precatalyst: 2.0 μmol, t = 30 min.
^{c}anilinium borate: 1.1 mmol [PhN(CH₃)₂H]⁺[B(C₆F₅)₄]⁻.
^{d}oligomeric products.
^{e}3 bar ethylene.

### Example 9

General Description of Ethylene Oligomerisation Experiments for Entries 22-24

The catalytic ethylene oligomerisation reactions were performed in a 250 mL glass autoclave (Buechi) in semi-batch mode (ethylene was added by replenishing flow to keep the pressure constant). The reactor was ethylene flow controlled and equipped with separated toluene, catalyst and co-catalyst injection systems.

During a oligomerisation run the pressure and the reactor temperature were kept constant while the ethylene flow was monitored continuously. In a typical semi-batch experiment, the autoclave was evacuated and heated for 1 h at 80° C. prior to use. The reactor was then brought to desired temperature, stirred at 1000 rpm and charged with 150 mL of toluene. After pressurizing with ethylene to reach 2 bar total pressure the autoclave was equilibrated for 10 min. Successive TEAL co-catalyst solution, activator (perfluorophenylborate), Diethyl aluminium chloride and 1 mL of a 0.001 M zirconium pre-catalyst stock solution in toluene was injected, to start the reaction. After the desired reaction time the reactor was vented and the residual aluminium alkyls were destroyed by addition of 50 mL of ethanol. Polymeric product was collected, stirred for 30 min in acidified ethanol and rinsed with ethanol and acetone on a glass frit. The polymer was initially dried on air and subsequently in vacuum at 80° C.

TABLE 4
Ethylene oligomerisation with Zr pre-catalyst IV in presence of
DEAC, TEAL co-catalyst and perfluorophenylborate activator.^{a}

| | TEAL | DEAC | M_{product} | Activity | Mₙ | |
|---|---|---|---|---|---|---|
| Entry | Al/Zr | Cl/Zr | [g] | [kg_{PE}mol_{cat}⁻¹h⁻¹bar⁻¹] | [kgmol⁻¹] | M_{w}/Mₙ |
| 22 | 2000 | 1 | 8.64 | 17300 | 1790 | 1.5 |
| 23 | 2000 | 3 | 3.11 | 6230 | 950 | 1.6 |
| 24 | 2000 | 6 | 0.88 | 1750 | 630 | 1.5 |

^{a}Precatalyst IV: 1.0 μmol; ammonium borate: 2.2 μmol [R₂N(CH₃)H]⁺[B(C₆F₅)₄]⁻ (R = C₁₆H₃₃—C₁₈H₃₇), Zr/B = 1/1.1; toluene: 150 mL; T = 50° C., p = 2 bar; t = 15 min.

### Example S1 (In-Situ)

General Description of Ethylene Oligomerisation Experiments for Entries 25+26 (Table 5)

The catalytic ethylene oligomerisation reactions were performed in a 250 mL glass autoclave (Buechi) in semi-batch mode (ethylene was added by replenishing flow to keep the pressure constant). The reactor was ethylene flow controlled and equipped with separated toluene, catalyst and co-catalyst injection systems. During an oligomerisation run the pressure and the reactor temperature were kept constant while the ethylene flow was monitored continuously.

In a typical semi-batch experiment, the autoclave was evacuated and heated for ½ h at 80° C. prior to use. The reactor was then brought to desired temperature, stirred at 500 rpm and charged with 150 mL of toluene. After pressurizing with ethylene to reach 2 bar total pressure the autoclave was equilibrated for 10 min. Successive chain transfer agent, activator, and 1 mL of a 0.001 M pre-catalyst stock solution in toluene was injected, to start the reaction. After 15 min reaction time the reactor was vented and the residual CSA alkyls were destroyed by addition of 20 mL of ethanol. Polymeric product was collected by filtration at 50° C., washed with acidified ethanol and rinsed with ethanol and acetone on a glass frit. The polymer was initially dried on air and subsequently in vacuum at 50° C. The soluble residue was analyzed by GC and/or GC-MS.

### Example S2 (In-Situ): General Description of Ethylene Oligomerisation Experiments for Entries 27+28 (Table 5)

The catalytic ethylene oligomerisation reactions were performed in a 250 mL glass autoclave (Buechi) in semi-batch mode (ethylene was added by replenishing flow to keep the pressure constant). The reactor was ethylene flow controlled and equipped with separated toluene, catalyst and co-catalyst injection systems. During a oligomerisation run the pressure and the reactor temperature were kept constant while the ethylene flow was monitored continuously. In a typical semi-batch experiment, the autoclave was evacuated and heated for ½ h at 80° C. prior to use. The reactor was then brought to desired temperature, stirred at 500 rpm and charged with 150 mL of toluene. After pressurizing with ethylene to reach 2 bar total pressure the autoclave was equilibrated for 10 min. Successive chain transfer agent, activator and chain displacement catalyst, all dissolved in toluene, were injected, to start the reaction. After 15 min reaction time the reactor was vented and the residual CSA alkyls were destroyed by addition of 20 mL of ethanol. The toluene solution was analyzed by GC and/or GC-MS.

### Example S3 (In-Situ): General Description of Ethylene Oligomerisation Experiments for Runs 29-38 (Table 6)

The catalytic ethylene oligomerisation reactions were performed in a 250 mL glass autoclave (Buechi) in semi-batch mode (ethylene was added by replenishing flow to keep the pressure constant). The reactor was ethylene flow controlled and equipped with separated toluene, catalyst and co-catalyst injection systems.

During a oligomerisation run the pressure and the reactor temperature were kept constant while the ethylene flow was monitored continuously. In a typical semi-batch experiment, the autoclave was evacuated and heated for ½ h at 80° C. prior to use. The reactor was then brought to desired temperature, stirred at 500 rpm and charged with the desired amount of toluene. After pressurizing with ethylene to reach the desired total pressure the autoclave was equilibrated for 10 min. Successive chain transfer agent, activator, chain displacement catalyst and pre-catalyst, all dissolved in toluene, were injected, to start the reaction. After the appropriate reaction time the reactor was vented and the residual CSA alkyls were destroyed by addition of 20 mL of ethanol. Polymeric product was collected by filtration at 50° C., washed with acidified ethanol and rinsed with ethanol and acetone on a glass frit. The polymer was initially dried on air and subsequently in vacuum at 50° C. The soluble residue was analyzed by GC and/or GC-MS.

### Example S4 (In-Situ)

The catalytic ethylene oligomerisation reaction was performed in a 1000 mL glass autoclave (Buechi) in semi-batch mode (ethylene was added by replenishing flow to keep the pressure constant). The reactor was ethylene flow controlled and equipped with separated toluene, catalyst and co-catalyst injection systems. During the oligomerisation run the pressure and the reactor temperature were kept constant while the ethylene flow was monitored continuously. In a typical semi-batch experiment, the autoclave was evacuated and heated for ½ h at 100° C. prior to use. The reactor was then brought to desired temperature, stirred at 500 rpm and charged with 300 mL toluene. After pressurizing with ethylene to reach the desired total pressure the autoclave was equilibrated for 10 min. 1000 μmol TEAL, 4 μmol of 2,3-Bis(2,6-diisopropylphenyl)-1,1-diethylguanidinato trimethanido zirconium(IV), 6 μmol of Dimethylaniliniumborate and 8 μmol of Bis(cyclooctadienyl)nickel(0), was added to start the reaction. 30 g of ethylene was dosed into the reactor.

The temperature was maintained at 60° C. After the appropriate reaction time the reactor was vented and the residual TEAL was destroyed by addition of 20 mL of ethanol. A sample is taken from the solution and analyzed via GC with nonane as internal standard.

TABLE 5
Comparative ethylene oligomerisation with GuaTiMe₃ (I) and GuaZrMe₃ (IV) precatalysts or Ni(stea)₂ and Ni(COD)₂ CDC's only.^{a}

| Entry | precat. [μmol] | CSA [μmol] | CDC [μmol] | C2 sump. [I] | con- yield [g] | Activity $[ \frac{{Kg}_{PE}}{{mol}_{cat} \cdot h \cdot bar} ]$ | m(C4) [g] | m(C6) [g] | m(C8) [g] | m(C10) [g] | m(C12) [g] | m_{solid} [g] | Mₙ $[ \frac{g}{mol} ]$ | M_{w}/ Mₙ |
|---|---|---|---|---|---|---|---|---|---|---|---|---|---|---|
| 25 | I (Ti)* | 10000 | — | 1.92 | 3.36 | 3360 | — | — | — | — | — | 1.92 | 1120 | 1.6 |
| | 2 | | | | | | | | | | | | | |
| 26 | IV (Zr) | 1000 | — | 12.14 | 15.18 | 30360 | — | — | — | — | — | 15.18 | 2480 | 1.9 |
| | 1 | | | | | | | | | | | | | |
| 27 | — | 1000 | Ni(stea)₂ | 0.00 | 0 | 0 | — | — | — | — | — | — | — | — |
| | | | 2 | | | | | | | | | | | |
| 28 | — | 1000 | Ni(COD)₂ | 0.10 | 0.12 | 125 | 0.10 | — | — | — | — | — | — | — |
| | | | 2 | | | | | | | | | | | |

^{a}activator: [Me₂NPhH][B(C₆F₅)₄], Zr/B = 1/2; toluene: 150 mL; p_{ethylene} = 2 bar; t = 15 min; *2,3-Bis(2,6-diisopropylphenyl)-1,1-diethylguanidinato trimethanido titanium(IV) (GuaTiMe3, I, J. Obenauf, W. P. Kretschmer, R. Kempe, Eur. J. Inorg. Chem. 2014, 1446-1453) was prepared according to published procedures.

TABLE 6
Ethylene oligomerisation with GuaTiMe₃ (I) and GuaZrMe₃ (IV) precatalysts in presence of CDC's.^{a}

| Entry | precat. [μmol] | CSA [μmol] | CDC [μmol] | C2 sump. [I] | con- yield [g] | Activity $[ \frac{{Kg}_{PE}}{{mol}_{cat} \cdot h \cdot bar} ]$ | m(C6) [g] | m(C8) [g] | m(C10) [g] | m(C12) [g] | m(C14) [g] | m_{solid} [g] | Mₙ $[ \frac{g}{mol} ]$ | M_{w}/ Mₙ |
|---|---|---|---|---|---|---|---|---|---|---|---|---|---|---|
| 29 | I (Ti)* | 10000 | Ni(COD)₂ | 6.68 | 8.35 | 8350 | — | — | — | — | — | 6.33 | 840 | 1.5 |
| | 2 | | 2 | | | | | | | | | | | |
| 30 | IV (Zr) | 1000 | Ni(acac)₂ | 10.40 | 13.00 | 26000 | 1.64 | 1.42 | 1.20 | 1.01 | 0.83 | 0.18 | | |
| | 1 | | 7.8 | | | | | | | | | | | |
| 31 | IV (Zr) | 1000 | Ni(stea)₂ | 10.77 | 13.46 | 26925 | 0.42 | 0.43 | 0.43 | 0.44 | 0.45 | 6.90 | 810 | 1.6 |
| | 1 | | 1 | | | | | | | | | | | |
| 32^{b} | IV (Zr) | 1000 | Ni(stea)₂ | 17.50 | 21.88 | 21875 | 1.67 | 1.58 | 1.50 | 1.38 | 1.19 | 1.69 | 740 | 1.5 |
| | 1 | | 2 | | | | | | | | | | | |
| 33^{b} | IV (Zr) | 1000 | Ni(COD)₂ | 17.23 | 21.54 | 21540 | 1.37 | 1.32 | 1.24 | 1.08 | 0.97 | 4.88 | 790 | 1.5 |
| | 1 | | 1 | | | | | | | | | | | |
| 34^{b} | IV (Zr) | 520 | Ni(COD)₂ | 15.20 | 19.00 | 19000 | 0.72 | 0.72 | 0.72 | 0.71 | 0.70 | 0.22 | 370 | 41.5^{g} |
| | 1 | | 2 | | | | | | | | | | | |
| 35^{b} | IV (Zr) | 330 | Ni(COD)₂ | 15.10 | 18.88 | 18875 | 1.01 | 0.99 | 0.98 | 0.92 | 0.86 | 0.40 | 520 | 133.7^{g} |
| | 1 | | 2 | | | | | | | | | | | |
| 36^{b} | IV (Zr) | 300 | Ni(COD)₂ | 15.00 | 18.75 | 18750 | 1.04 | 1.02 | 0.98 | 0.94 | 0.87 | 1.95 | 390 | 2.3 |
| | 1 | | 2 | | | | | | | | | | | |
| 37^{c} | IV (Zr) | 280 | Ni(COD)₂ | 16.80 | 21.00 | 21000 | 0.91 | 0.91 | 0.92 | 0.91 | 0.89 | 8.34 | 390 | 2.0 |
| | 1 | | 2 | | | | | | | | | | | |
| 38^{d} | IV (Zr) | 650 | Ni(COD)₂ | 0.80 | 1.50 | 3000 | 0.36 | 0.26^{e} | — | 0.16 | 0.10^{f} | 0.05 | n.d. | n.d. |
| | 1 | | 2 | | | | | | | | | | | |

^{a}activator: [Me₂NPhH][B(C₆F₅)₄], Zr/B = 1/2; toluene: 150 mL; p_{ethylene} = 2 bar; t = 15 min.
^{b}t = 30 min, CSA = . Yield was calculated from ethylene consumption, m(C4) was not determined.
^{c}p_{ethylene} = 4 bar.
^{d}p_{propylene} = 2 bar.
^{e}Cg.
^{f}C₁₅.
^{g}bimodal;* 2,3-Bis(2,6-diisopropylphenyl)-1,1-diethylguanidinato trimethanido titanium(IV) (GuaTiMe3, I, J. Obenauf, W. P. Kretschmer, R. Kempe, Eur. J. Inorg. Chem. 2014, 1446-1453) was prepared according to published procedures.

Waxy product was collected by filtration (0.2 μm) at 50° C., washed with acidified ethanol and rinsed with ethanol and acetone on a glass frit. The filtrate was initially dried on air and subsequently in vacuum at 50° C. and analyzed via GPC. The permeate was analyzed by GC and/or GC-MS.

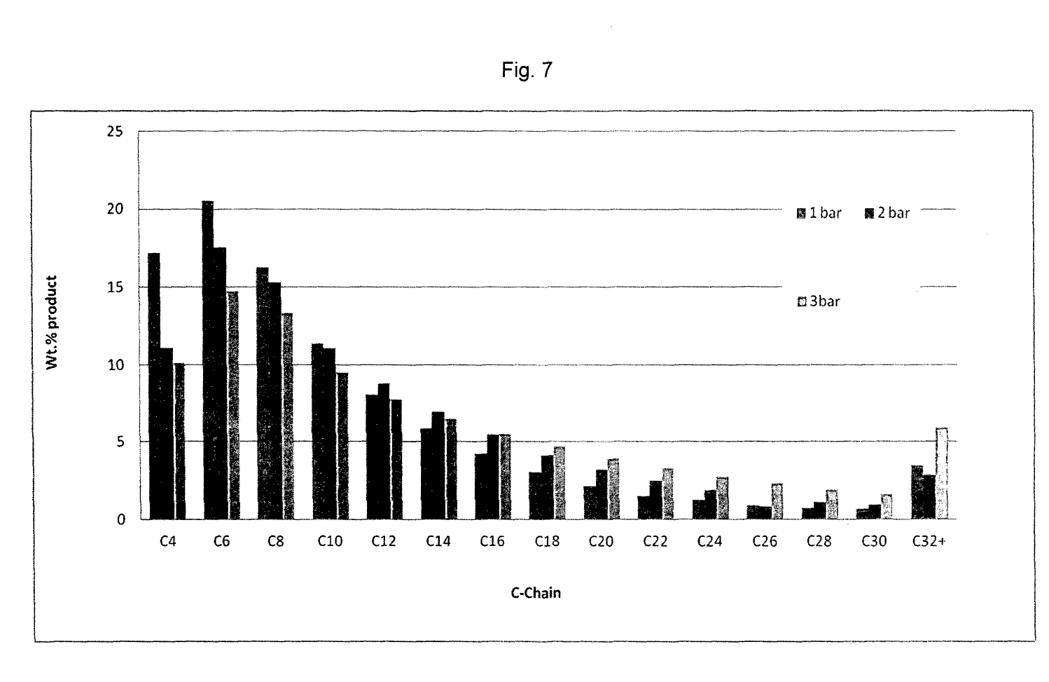

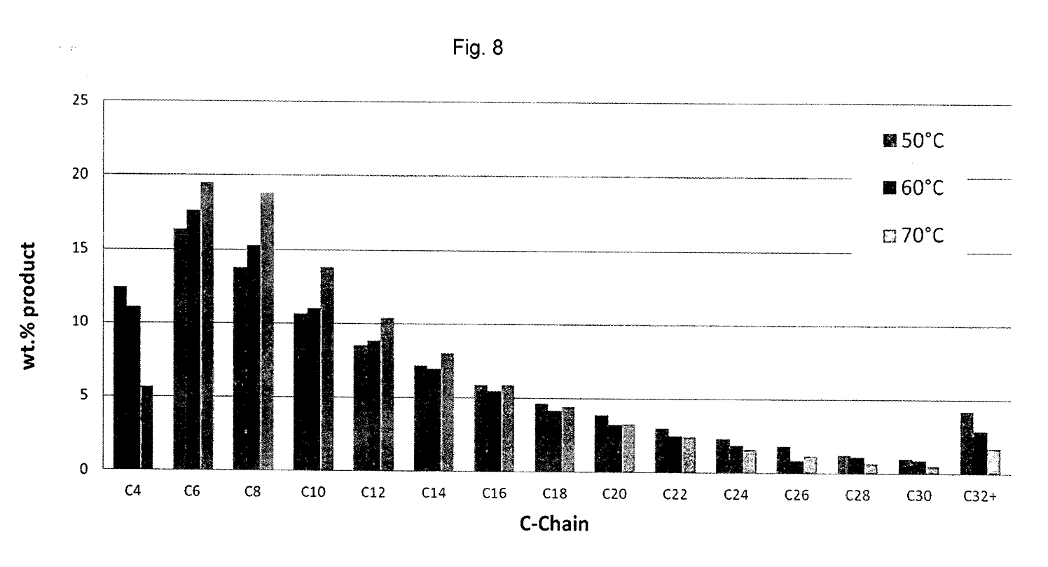

The effect of applied pressure and temperature are shown in FIGS. 7 and 8.

### Example S5 (Sequential Process)

The catalytic ethylene oligomerisation reaction was performed in a 1000 mL glass autoclave (Buechi) in semi-batch mode (ethylene was added by replenishing flow to keep the pressure constant). The reactor was ethylene flow controlled and equipped with separated toluene, catalyst and co-catalyst injection systems. During a oligomerisation run the pressure and the reactor temperature were kept constant while the ethylene flow was monitored continuously. In a typical semi-batch experiment, the autoclave was evacuated and heated for ½ h at 100° C. prior to use. The reactor was then brought to desired temperature, stirred at 500 rpm and charged with 300 mL toluene. After pressurizing with ethylene to reach the desired total pressure the autoclave was equilibrated for 10 min. 40000 μmol TEAL, 4 μmol of 2,3-Bis(2,6-diisopropylphenyl)-1,1-diethylguanidinato trimethanido zirconium(IV), 4 μmol of trioctylammonium borate was added to start the reaction. 22 g of ethylene was dosed into the reactor and the temperature was maintained at 60° C.

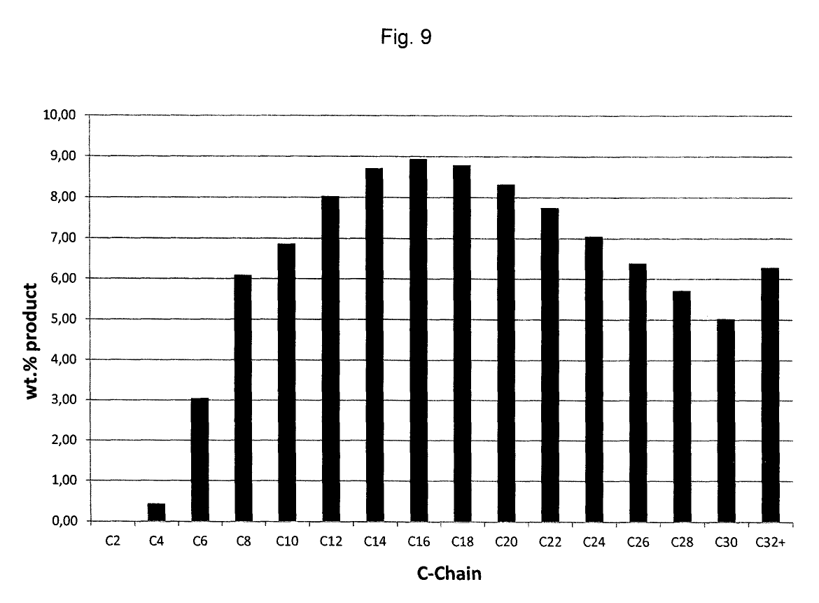

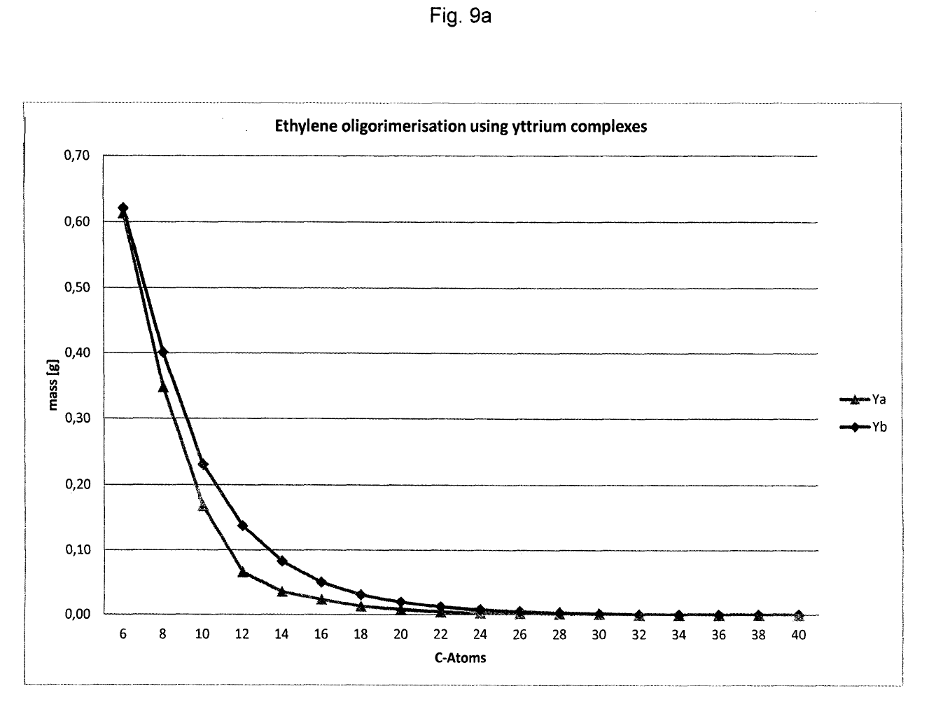

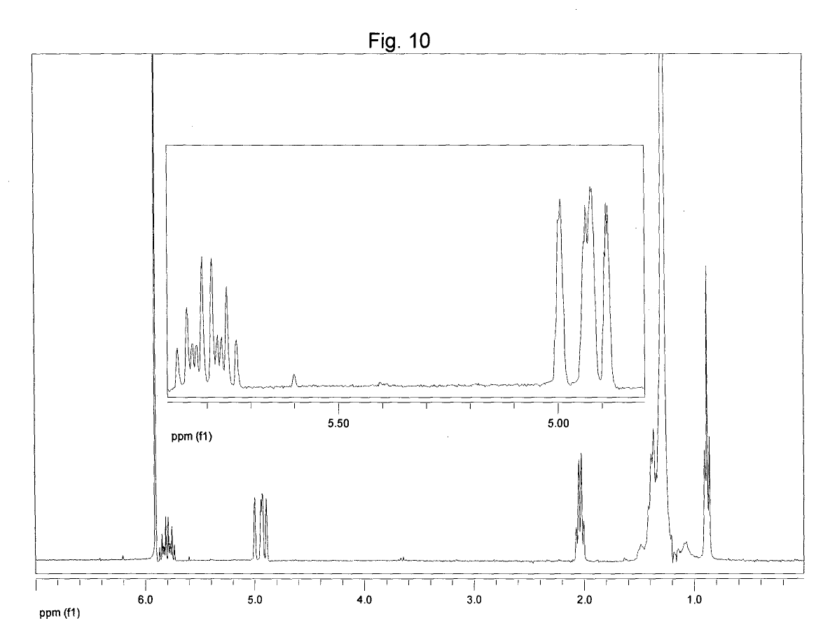

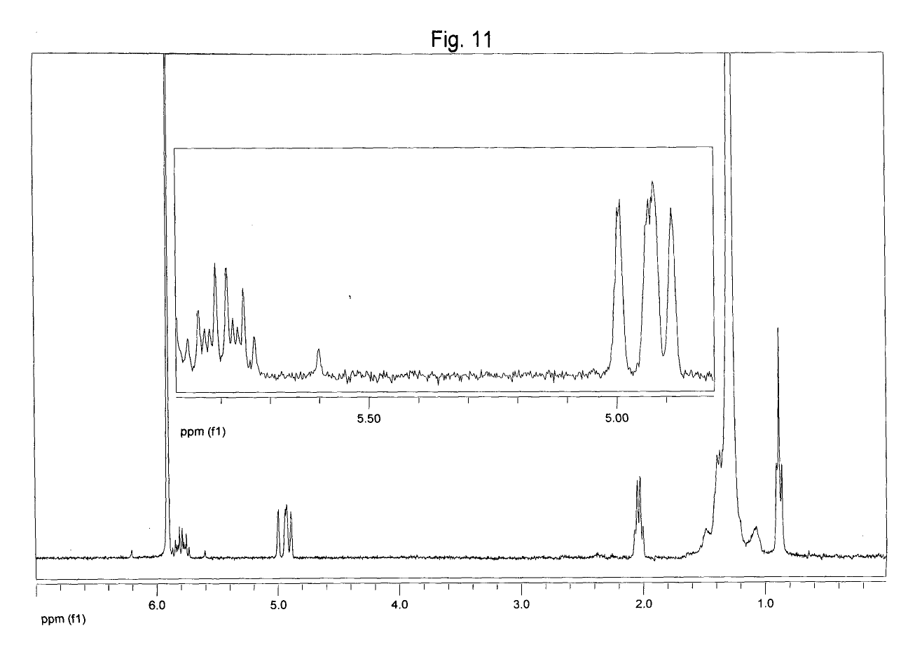

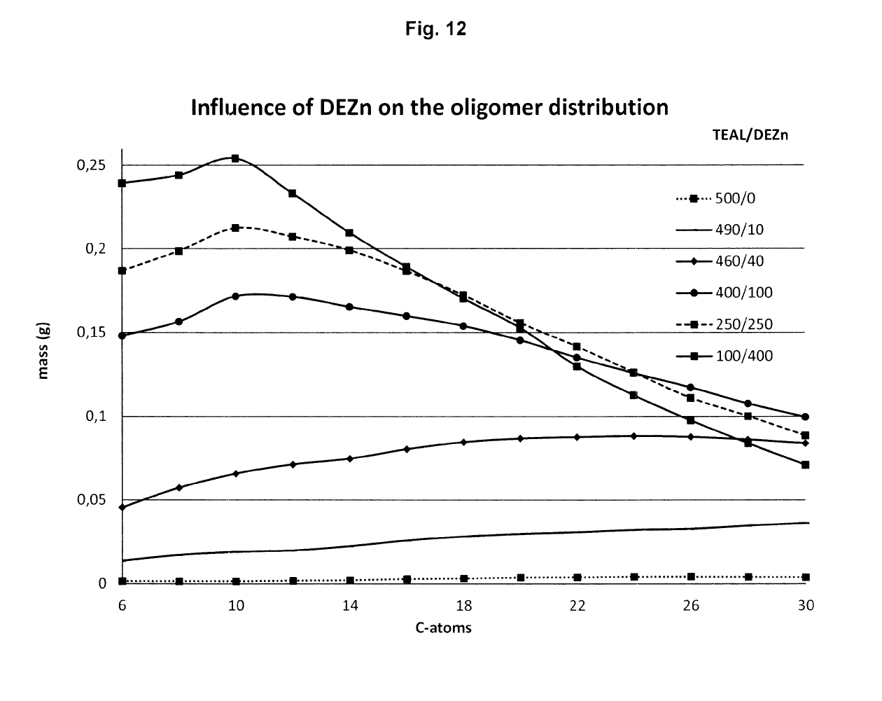

After the desired amount of ethylene was consumed the reactor was depressurized and the reactor flushed with argon. Subsequently the temperature was raised to 100° C. for 1 hour. 8 μmol Bis(cyclooctadienyl)nickel(0), was added to the reactor via a syringe. A temperature of 120° C. was set and maintained via a thermostat. The reactor was pressurized with ethylene again and the reaction monitored until no more ethylene was consumed. The residual TEAL was destroyed by addition of 20 mL of ethanol. A sample was taken from the solution and analyzed via GC with nonane as internal standard. Waxy product was collected by filtration (0.2 μm) at 50° C., washed with acidified ethanol and rinsed with ethanol and acetone on a glass frit. The filtrate was initially dried on air and subsequently in vacuum at 50° C. and analyzed via GPC. The permeate was analyzed by GC and/or GC-MS. The distribution of the obtained linear α-olefins are shown in FIG. 9.

### Example S6 (In-Situ): Single Chain Shuttling Using Yttrium Complexes as CCTP Catalysts (Table 7)

The following yttrium pre-catalysts were employed in the ethylene oligorimerisation experiments:

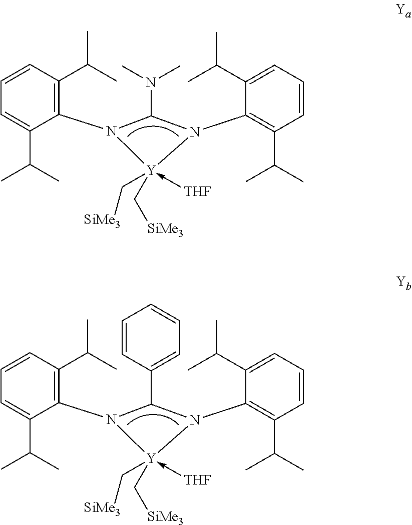

The catalytic ethylene oligomerization reactions were performed in an 800 mL autoclave (Buechi) in semi-batch mode (ethylene was added by replenishing flow to keep the pressure constant). The reactor was ethylene flow controlled and equipped with separated toluene, catalyst and co-catalyst injection systems. During an oligomerization run the pressure and the reactor temperature were kept constant while the ethylene flow was monitored continuously. In a typical semi-batch experiment, the autoclave was evacuated and heated for ½ h at 80° C. prior to use. The reactor was then brought to 80° C., stirred and charged with 250 mL of toluene. After pressurizing with ethylene to reach the 5 bar the autoclave was equilibrated for 3 min. Successive TEAL co-catalyst solution, activator (methyldialkylammonium-tetrakis(pentafluorophenyl)borate) and after an additional equilibration yttrium pre-catalyst stock solution in toluene was injected, to start the reaction. After the 15 min the reactor was vented and the residual TEAL was destroyed by addition of 20 mL of ethanol. A sample was taken from the solution and analyzed via GC with cumene as internal standard. Waxy product was collected by filtration (0.2 μm) at 50° C., washed with acidified ethanol and rinsed with ethanol and acetone on a glass frit. The permeate was analyzed by GC and/or GC-MS. The distribution of the obtained linear oligomerised alpha olefins are shown in FIG. 9a.

TABLE 7
Ethylene oligomerisation with Y pre-catalysts Ya and Yb,
TEAL co-catalyst and
methyldialkylammoniumtetrakis(pentafluorophenyl)borate activator.^{a}

| Entry | Catalyst | n(Al(Et)₃) [μmol] | n(Ni(cod)₂) [μmol] | activity $[ \frac{kg}{mol \cdot h \cdot bar} ]$ |
|---|---|---|---|---|
| 39 | Ya | 500 | 2 | 128 |
| 40 | Yb | 500 | 2 | 175 |

^{a}Pre-catalyst: 10.0 μmol; ammonium borate: 10.0 μmol [R₂N(CH₃)H]⁺[B(C₆F₅)₄]⁻ (R = C₁₆H₃₃-C₁₈H₃₇); Y/B = 1/1; CDC 2 μmol Ni(COD)₂; toluene: 250 mL; T = 80° C., p = 5 bar.

Experimental Section Dual Chain Shuttling

N,N,N-trialkylammonium (tetrapentafluorophenyl)borate ([R₂NMeH][B(C₆F₅)₄], R═C₁₆H₃₃—C₁₈H₃₇, 6.2 wt-% B(C₆F₅)₄ in Isopar, DOW Chemicals), Bis(1,5-cyclooctadiene)nickel(0), and Zirconium(IV)chloride are commercially available from abcr GmbH & Co. KG. Triethyl aluminum (SASOL Germany GmbH) and Diethyl zinc (15 wt-% in toluene, Sigma-Aldrich) were used as received. The ligand precursor N-(2,6-diisopropylphenyl)pyridine-2-amine (A. Noor, W. P. Kretschmer, R. Kempe, Eur. J. Inorg. Chem. 2006, 2683), 6-Chloro-N-(2,6-diisopropylphenyl)pyridin-2-amine (M. Hafeez, W. P. Kretschmer, R. Kempe, Eur. J. Inorg. Chem. 2011, 5512-5522) and the metal precursor (1,3-di-tert-butylcyclopenta-1,3-dienyl)-trimethanidozirconium(IV) (J. Amor, T. Cuenca, M. Galakhov, P. Royo, J. Organomet. Chem 1995, 497, 127-131) were prepared according to published procedures.

### Synthesis of Pre-Catalyst 1

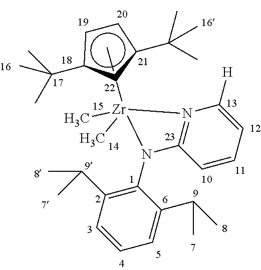

### Synthesis of (1,3-di-tert-butylcyclopenta-1,3-dienyl)-(N-(2,6-diisopropylphenyl)pyridin-2-amidinato)-dimethanidozirconium(IV) (I)

To a solution of ApH (88 mg, 0.35 mmol) in benzene (0.5 mL) was added (1,3-di-tert-butylcyclopenta-1,3-dienyl)-trimethanidozirconium(IV) (109 mg, 0.35 mmol). The mixture was shaken for 15 min, until the formation of methane gas was finished, filtrated and used without further purification. NMR spectroscopic analysis showed an almost quantitative formation of the desired complex II. ¹H NMR (300 MHz, C₆D₆):δ=0.47 [s, 6H, H^{14,15}], 1.07 (d, J=6.6 Hz, 6H, H^{7,8}), 1.13 (s, 18H, H^{16,16′}), 1.32 (d, J=6.6 Hz), 3.44 (sept, J=6.6 Hz, 2H, H^{9,9′}), 5.56 (d, J=8.8 Hz, 1H, H³), 5.82 (m, 1H, H⁵), 5.99 (d, J=2.2 Hz, 2H, H^{19,20}), 6.45 (t, J=2.3 Hz, 1H, H²²), 6.67 (t, J=7.3 Hz, 1H, H⁴), 6.98-7.31 (m, 3H, H^{10,11,12}), 7.48 (d, J=5 Hz, 1H, H²⁴). ¹³C NMR (300 MHz, C₆D₆):δ=24.68 (s, 2C, C^{7′,8′}), 26.05 (s, 2C, C^{7,8}), 28.66 (s, 2C, C^{9;9′}), 31.97 (s, 6C, C^{16;16′}), 33.26 (s, 2C, C^{14,15}), 107.55 (s, 1C, C³), 108.37 (s, 2C, C^{19,20}), 109.67, 109.74, 124.07, 124.88, 126.04, 126.33, 128.91 (s, 10C, C^{1,2,4,6,18,21,22}), 129.67, 140.21, 141.63, 144.08, 145.01 (s, 5C, C^{10,11,12,13,23})

### Synthesis of Pre-Catalyst II

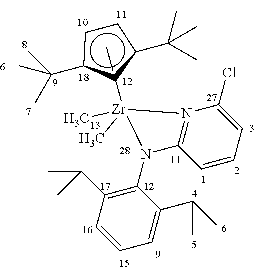

### Synthesis of (1,3-di-tert-butylcyclopenta-1,3-dienyl)-(6-chloro-N-(2, 6-diisopropylphenyl)pyridin-2-amidinato)-dimethanidozirconium(IV) (II)

To a solution of Ap^{Cl}H (45 mg, 0.15 mmol) in benzene (0.5 mL) was added (1,3-di-tert-butylcyclopenta-1,3-dienyl)-trimethanidozirconium(IV) (49 mg, 0.15 mmol). The mixture was shaken for 15 min, until the formation of methane gas was finished, filtrated and used without further purification. NMR spectroscopic analysis showed an almost quantitative formation of the desired complex III. ¹H NMR (300 MHz, C₆D₆):δ=0.60 [s, 6H, H^{14,15}], 1.01 [d, J=6.4 Hz, 6H, H^{7,8}], 1.17 [s, 18H, H^{16,16′}], 1.29 [d, J=7.0 Hz, 6H, H^{7′,8′}], 3.46 [sept, J=6.5 Hz, 2H, H^{9,9′}], 5.33 [d, J=8.2 Hz, 1H, H³], 5.88 [d, J=6.3 Hz, 1H, H⁵ ], 6.29 [d, J=2.9 Hz, 2H, H^{19,20}] 6.3 [t, J=8.2 Hz, 1H, H²²], 6.62 [t, J=2.6 Hz, 1H, H²²], 6.95-7.19 [m, 3H, H^{10,11,12}].

¹³C NMR (300 MHz, C₆D₆):δ=24.48 (s, 2C, C^{7′,8′}), 26.04 (s, 2C, C^{7,8}), 28.80 (s, 2C, C^{9;9′}), 32.70 (s, 6C, C^{16;16′}), 45.58 (s, 2C, C^{14,15}), 105.58 (s, 1C, C³), 108.51 (s, 2C, C^{19,20}), 110.20 (s, 1C, C⁴), 111.23, 125.02, 128.07, 128.92, 129.67, 141.34, 142.43, 143.31, 144.67, 147.40, (s, 10C, C^{1,2,6,18,21,22,10,11,12,13}), 171.87 (s, 1C, C²³). CHN anal. C₃₂H₄₆Cl₁N₂Zr (585.40): C, 65.65; H, 7.92; N, 4.79. Found: C, 65.94, H, 8.21, N, 4.45.

Examples for Dual Chain Shuttling

General Description of Ethylene Oligomerisation Experiments for Runs D1 to D12 (Table 8)

The catalytic ethylene oligomerization reactions were performed in a 300 mL glass autoclave (Buechi) in semi-batch mode (ethylene was added by replenishing flow to keep the pressure constant). The reactor was ethylene flow controlled and equipped with separated toluene, catalyst and co-catalyst injection systems. During an oligomerization run the pressure and the reactor temperature were kept constant while the ethylene flow was monitored continuously. In a typical semi-batch experiment, the autoclave was evacuated and heated for ½ h at 80° C. prior to use. The reactor was then brought to desired temperature, stirred at 1000 rpm and charged with the desired amount of toluene. After pressurizing with ethylene to reach the desired total pressure the autoclave was equilibrated for 3 min. Successive chain transfer agent, activator, chain displacement catalyst and pre-catalyst, all dissolved in toluene, were injected, to start the reaction. After the appropriate reaction time the reactor was vented and the residual CSA alkyls were destroyed by addition of 20 mL of ethanol. Solid product was collected by filtration at 50° C., washed with acidified ethanol and rinsed with ethanol and acetone on a glass frit. The wax was initially dried on air and subsequently at 50° C. The soluble residue was analyzed by GC and/or GC-MS.

TABLE 8
Ethylene oligomerisation examples with Cp”ApZrMe₂ (I) and Cp”Ap^{Cl}ZrMe₂ (II) precatalysts, Ni(COD)₂ as CDC and
DEZn/TEAL mixtures as CSA.^{a}

| Entry | precat. (M) [μmol] | CSA [μmol] | time [min] | C2 con- sump. [I] | yield [g] | Activity $[ \frac{{Kg}_{PE}}{{mol}_{cat} \cdot h \cdot bar} ]$ | m(C6) [g] | m(C8) [g] | m(C10) [g] | m(C12) [g] | m(C14) [g] | m_{solid} [g] | Mₙ $[ \frac{g}{mol} ]$ | M_{w}/ Mₙ |
|---|---|---|---|---|---|---|---|---|---|---|---|---|---|---|
| D1 | I | 500 | 12.0 | 2 | 2.50 | 2940 | 0.014 | 0.015 | 0.017 | 0.020 | 0.022 | 1.77 | 1380 | 1.4 |
| | (Ap, 2) | (10/490) | | | | | | | | | | | | |
| D2 | I | 500 | 11.3 | 2 | 2.50 | 3320 | 0.046 | 0.057 | 0.066 | 0.072 | 0.075 | 1.12 | 920 | 1.2 |
| | (Ap, 2) | (40/460) | | | | | | | | | | | | |
| D35 | I | 500 | 9.4 | 2 | 2.50 | 3990 | 0.112 | 0.120 | 0.129 | 0.122 | 0.116 | — | — | — |
| | (Ap, 2) | (100/400) | | | | | | | | | | | | |
| D46 | I | 500 | 9.9 | 2 | 2.50 | 3800 | 0.196 | 0.208 | 0.200 | 0.185 | 0.169 | 0.39 | 377 | 1.1 |
| | (Ap, 2) | (250/250) | | | | | | | | | | | | |
| D5 | I | 500 | 11.1 | 2 | 2.50 | 3380 | 0.239 | 0.244 | 0.254 | 0.233 | 0.210 | — | — | — |
| | (Ap, 2) | (400/100) | | | | | | | | | | | | |
| D6 | II | 300 | 30 | 2.81 | 3.52 | 1760 | 0.012 | 0.014 | 0.015 | 0.018 | 0.021 | 2.85 | 1460 | 1.4 |
| | (Ap^{Cl}, 2) | (50/250) | | | | | | | | | | | | |
| D7 | II | 300 | 30 | 2.99 | 3.74 | 1870 | 0.028 | 0.030 | 0.035 | 0.042 | 0.047 | 2.68 | 1050 | 1.5 |
| | (Ap^{Cl}, 2) | (100/200) | | | | | | | | | | | | |
| D8 | II | 300 | 30 | 3.11 | 3.89 | 1950 | 0.068 | 0.081 | 0.089 | 0.098 | 0.105 | 1.71 | 870 | 1.3 |
| | (Ap^{Cl}, 2) | (150/150) | | | | | | | | | | | | |
| D9 | II | 300 | 30 | 2.30 | 2.88 | 1440 | 0.041 | 0.053 | 0.058 | 0.065 | 0.071 | 1.31 | 1000 | 1.2 |
| | (Ap^{Cl}, 2) | (200/100) | | | | | | | | | | | | |
| D10 | II | 300 | 30 | 1.82 | 2.28 | 1140 | 0.062 | 0.074 | 0.081 | 0.088 | 0.092 | 0.53 | 950 | 1.9 |
| | (Ap^{Cl}, 2) | (250/50) | | | | | | | | | | | | |
| D11^{b} | II | 300 | 30 | 1.74 | 2.18 | 1080 | 0.049 | 0.062 | 0.061 | 0.066 | 0.070 | 0.61 | 1010 | 1.3 |
| | (Ap^{Cl}, 2) | (150/150) | | | | | | | | | | | | |
| D12 | II | 500 | 30 | 1.72 | 2.15 | 1080 | 0.121 | 0.124 | 0.134 | 0.139 | 0.138 | 0.02 | 840 | 2.2 |
| | (Ap^{Cl}, 2) | (250/250) | | | | | | | | | | | | |

^{a}toluene: 150 mL; CDC: 2 μmol Ni(COD)₂; activator: 2.2 μmol [R₂NMeH][B(C₆F₅)₄] (R = C₁₆H₃₃-C₁₈H₃₇), Zr/B = 2/2.2; p_{ethylene} = 2 bar; t = 15 min.
^{b}CDC: 24 μmol Ni(COD)₂.

1. Process for the manufacture of oligomerised olefins
i) by bringing in contact with each other
(a) one or more C2 to C8 olefins,
(b) a coordinate chain transfer polymerization catalyst (CCTP catalyst) comprising one or more organo metallic transition metal compounds and one or more ligands,
(c) a chain shuttling agent (CSA) being one or more metal alkyls selected from the group II, XII and XIII,
(d) a chain displacement catalyst (CDC) being one or more member selected from the group consisting of a nickel salt, a cobalt salt, an organo metallic nickel complex and an organo metallic cobalt complex,
or
ii) by bringing in contact with each other (a), (b), (c) and (d), wherein (c) is
(c-1) the chain shutting agent (CSA) comprises two metal alkyls one being one or more Zn alkyl compounds (CSA(1)) the other being one or more XIII metal alkyls (CSA(2)),
to form a growth composition thereby obtaining oligomerised olefins having an oligomerisation degree of 2 to 100,
wherein for process i) (d) is brought in contact only at a later point in time with the growth composition when the oligomerisation has commenced or has come to an end and (b) is at least partially or completely transformed into an inactive reaction product or inactive degradation product.

2. The process according to claim 1, wherein the growth composition further comprises an activator for the coordinative chain transfer polymerization catalyst (CCTP catalyst) being an aluminium or boron containing compound comprising at least one hydrocarbyl group.

3. The process according to claim 1, wherein the olefin is one or more member selected from the group consisting of ethylene, propylene, 1-butene, 1-pentene and 1-hexene.

4. The process according to claim 1, wherein the one or more organo metallic transition metal compounds comprises one or two transition metals.

5. The process according to claim 1, wherein one or two ligands are selected from cyclopentadienyl, indenyl, fluorine, diamide ligands, phenoxy-imine-ligand, indolide-imine-ligands, amidinate, guanidinate, amidopyridine, pyrrdinimine and alcoholate each optionally substituted.

6. The process according to claim 1, wherein for process i) the CCTP catalyst is deactivated during or after the oligomerisation by heating the growth composition or by bringing the CCTP catalyst in contact with a catalyst poison.

7. The process according to claim 1 wherein the chain shuttling agent (CSA) is a C1 to C30 hydrocarbyl metal compound, methyl-alumoxane or both, the metal being aluminium, zinc, magnesium, indium or gallium.

8. The process according to claim 1 wherein the chain displacement catalyst (CDC) is selected from nickel halogenides, cobalt halogenides, nickel cyclooctadiene, cobalt cyclooctadiene, nickel acetylactonate, C1 to C30 carboxylic acid salts of nickel and mixtures thereof.

9. The process according to claim 2 wherein the activator is methyl aluminoxan, or a perfluorated aluminate or a boron containing compound or combinations thereof.

10. The process according to claim 1 wherein the molar ratio of the coordinative chain transfer polymerization catalyst (CCTP catalyst) to the chain shuttling agent (CSA) is 1:>50000 or 1:50 to 1:10000, except for methyl alumoxane as the CSA.

11. The process according to claim 1 wherein the molar ratio of the coordinative chain transfer polymerization catalyst (CCTP catalyst) to the chain displacement catalyst (CDC)
prior or during the oligomerisation is 1:0.5 to 1:50, and
after the oligomerisation in the concentration of the chain displacement catalyst (CDC) is between 1 to 10000 ppm (w/w) relative to the growth composition.

12. The process according to claim 2 wherein the molar ratio of the coordinative chain transfer polymerization catalyst (CCTP catalyst) to the activator is 1:1 to 1:4, except in case where methyl alumoxane acts as the activator.

13. The process according to claim 1 wherein the growth composition further comprises a liquid reaction medium, the liquid reaction medium comprising aromatic hydrocarbons.

14. The process according to claim 1 wherein the reaction is carried out at a C2 or C3 or C2 and C3 olefin pressure of 0.2 to 60 bar, or a C4 pressure of 0.2 to 20 bar.

15. The process according to claim 1, wherein the coordinative chain transfer polymerization catalyst comprises as transition metal Ti, Zr or Hf and one ligand per metal of the following formula 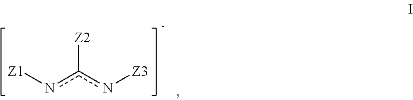
the ligand being bound to the metal, wherein
Z1, Z2 and Z3=are independently hydrocarbon or heteroatom containing hydrocarbon moieties, wherein the heteroatom, if present, for Z1 or Z3 is not directly adjacent to the N-atom and, wherein Z1, Z2 and Z3 independently from each other are optionally linked with one or more of each other.

16. The process according to claim 1, wherein the coordinative chain transfer polymerization catalyst comprises as transition metal Ti, Zr or Hf and one ligand per metal having the following sub-structural element: 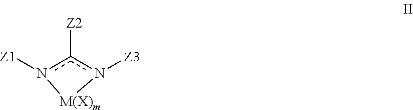
wherein
Z1, Z3=each are independently from each other a di-ortho substituted aromatic moiety,
each being independently hydrocarbon moieties or heteroatom containing hydrocarbon moieties, wherein the heteroatom, if present, is not directly adjacent to the N-atom,
Z2=is a hydrocarbon moiety or a heteroatom containing hydrocarbon moiety, Z1, Z2 and Z3 independently from each other are optionally linked with one or more of each other,
M=Titanium, Zirconium or Hafnium
X=halogen, hydrocarbyl, hydride; alkoxide, amide, optionally substituted and independent of each m, and
m=1 to 4.

17. The process of claim 15 wherein Z2 is NR1R2 with R1 and R2 independently from each other are C1 to C40 hydrocarbon moieties, optionally comprising one or more heteroatoms.

18. The process according to claim 1 wherein for process ii) in step (c-1) the chain shutting agent (CSA(2)) is an aluminium alkyl compound.

19. The process according to claim 18 wherein the process does not include that (d) is brought in contact with the growth composition only at a later point in time when the oligomerisation has commenced or has come to an end and (b) is at least partially or completely transformed into an inactive reaction product or inactive degradation product.

20. The process according to claim 18, wherein the metal has a hydrocarbyl-2-pyridyl amine ligand and an, optionally substituted, cyclopentadienyl ligand.

21. The process according to claim 18, wherein the molar ratio of the coordinative chain transfer polymerization catalyst (CCTP catalyst) to the chain shuttling agent (CSA(1)), being a zinc hydrocarbyl compound is 1/10 to 1/500.

22. The process according to claim 18, wherein the molar ratio of the coordinative chain transfer polymerization catalyst (CCTP catalyst) to the chain shuttling agent (CSA(2)), being an aluminium alkyl compound, is 1/50 to 1/500.

23. The process according to claim 18, wherein the molar ratio of the (CSA(1)) to the (CSA(2)) is 1/49 to 5/1.

24. The process according to claim 20, wherein the zirconium cyclopentadienyl hydrocarbyl-2-pyridyl amine alkyl compound is 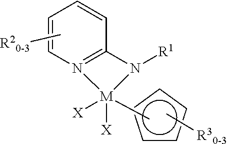
wherein
R1 and R2=independent from each other is hydrocarbyl, or halogen, wherein R2 is branched at the 2-position;
R3=is independently from each other zero to three hydrocarbyl
and
M=titanium, zirconium or hafnium.
X=independent of each other halogen; hydrocarbyl, C1 to C40, and alkysubstituted cyclopentadiene.
